SEQUENCE LISTING (27 sequences)

SEQ ID NO: 1 - protein, 234 aa, Mus musculus
EVKLVESGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSDIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKRA

SEQ ID NO: 2 - protein, 249 aa, Artificial Sequence
  Humanised scFv
EVKLVESGPELKKPGETVKISCKASGYTFTNYGMNWVKQAPGKGLKWMGWLNTYTGESIYPDDFKGRFAFSLETSASTAYLQINNLKSEDTATYFCARGDYGYDDPLDYWGQGTSVTVSSGGGGSGGGGSGGGGSDIVMTQAPPSVPVTPGESVSISCRSSKSLLHTNGNTYLHWFLQRPGQSPRLLIYRMSVLASGVPDRFSGSGSGTAFTLSISRVEAEDVGVYYCMQHLEYPLTFGAGTKLEIKRA

SEQ ID NO: 3 - protein, 5 aa, Artificial Sequence
  Humanised scFV heavy chain CDR1
NYGMN

SEQ ID NO: 4 - protein, 17 aa, Artificial Sequence
  Humanised scFV heavy chain CDR2
WLNTYTGESIYPDDFKG

SEQ ID NO: 5 - protein, 11 aa, Artificial Sequence
  Humanised scFV heavy chain CDR3
GDYGYDDPLDY

SEQ ID NO: 6 - protein, 16 aa, Artificial Sequence
  Humanised scFV light chain CDR1
RSSKSLLHTNGNTYLH

SEQ ID NO: 7 - protein, 7 aa, Artificial Sequence
  Humanised scFV light chain CDR2
RMSVLAS

SEQ ID NO: 8 - protein, 9 aa, Artificial Sequence
  Humanised scFV light chain CDR3
MQHLEYPLT

SEQ ID NO: 9 - protein, 17 aa, Artificial Sequence
  Linker sequence
SSGGGGSGGGGSGGGGS

SEQ ID NO: 10 - protein, 283 aa, Artificial Sequence
  Humanised scFv linked to Bivalirudin
EVKLVESGPELKKPGETVKISCKASGYTFTNYGMNWVKQAPGKGLKWMGWLNTYTGESIYPDDFKGRFAFSLETSASTAYLQINNLKSEDTATYFCARGDYGYDDPLDYWGQGTSVTVSSGGGGSGGGGSGGGGSDIVMTQAPPSVPVTPGESVSISCRSSKSLLHTNGNTYLHWFLQRPGQSPRLLIYRMSVLASGVPDRFSGSGSGTAFTLSISRVEAEDVGVYYCMQHLEYPLTFGAGTKLEIKRAGGGGSGGGGSGGGGFPRPGGGGNGDFEEIPEEYL

SEQ ID NO: 11 - protein, 328 aa, Artificial Sequence
  Humanised scFv linked to Lepirudin
EVKLVESGPELKKPGETVKISCKASGYTFTNYGMNWVKQAPGKGLKWMGWLNTYTGESIYPDDFKGRFAFSLETSASTAYLQINNLKSEDTATYFCARGDYGYDDPLDYWGQGTSVTVSSGGGGSGGGGSGGGGSDIVMTQAPPSVPVTPGESVSISCRSSKSLLHTNGNTYLHWFLQRPGQSPRLLIYRMSVLASGVPDRFSGSGSGTAFTLSISRVEAEDVGVYYCMQHLEYPLTFGAGTKLEIKRAGGGGSGGGGSGGGGLTYTDCTESGQNLCLCEGSNVCGQGNKCILGSDGEKNQCVTGEGTPKPQSHNDGDFEEIPEEYLQ

SEQ ID NO: 12 - protein, 259 aa, Artificial Sequence
  Humanised scFv
EVQLVESGPELKKPGETVKISCKASGYTFTNYGMNWVKQAPGKGLKWMGWLNTYTGESIYPDDFKGRFAFSLETSASTAYLQINNLKSEDTATYFCARGDYGYDDPLDYWGQGTSVTVSSGGGGSGGGGSGGGGSDIVMTQAPPSVPVTPGESVSISCRSSKSLLHTNGNTYLHWFLQKPGQSPRLLIYRMSVLASGVPDRFSGSGSGTDFTLKISRVEAEDVGVYYCMQHLEYPLTFGAGTKLEIKRAEQKLISEEDL

SEQ ID NO: 13 - protein, 118 aa, Artificial Sequence
  Humanised ScFv heavy chain variable region
EVQLVESGPELKKPGETVKISCKASGYTFTNYGMNWVKQAPGKGLKWMGWLNTYTGESIYPDDFKGRFAFSLETSASTAYLQINNLKSEDTATYFCARGDYGYDDPLDYWGQGTSVTV

SEQ ID NO: 14 - protein, 124 aa, Artificial Sequence
  Humanised ScFv light chain variable region
DIVMTQAPPSVPVTPGESVSISCRSSKSLLHTNGNTYLHWFLQKPGQSPRLLIYRMSVLASGVPDRFSGSGSGTDFTLKISRVEAEDVGVYYCMQHLEYPLTFGAGTKLEIKRAEQKLISEEDL

SEQ ID NO: 15 - protein, 258 aa, Artificial Sequence
  scFv
EVQLEESGGGLVKPGGSLKLSCAASGFTFSTYAMSWVRQTPEKRLEWVATISSGGGYTYYPDSVKGRFTISRDNAKNTLYLQMSSLRSEDSAMYYCARIYYYGSRNGYFDHWGQGTTLTVSSGGGGSGGGGSGGGGSDIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKEIKRADAAPTVSS

SEQ ID NO: 16 - protein, 7 aa, Artificial Sequence
  scFv heavy chain CDR1
FSTYAMS

SEQ ID NO: 17 - protein, 17 aa, Artificial Sequence
  scFv heavy chain CDR2
TISSGGGYTYYPDSVKG

SEQ ID NO: 18 - protein, 14 aa, Artificial Sequence
  scFv heavy chain CDR3
RIYYYGSRNGYFDH

SEQ ID NO: 19 - protein, 15 aa, Artificial Sequence
  scFv light chain CDR1
RASKSVSTSGYSYMH

SEQ ID NO: 20 - protein, 8 aa, Artificial Sequence
  scFv light chain CDR2
YLVSNLES

SEQ ID NO: 21 - protein, 9 aa, Artificial Sequence
  scFv light chain CDR3
QHIRELTRS

SEQ ID NO: 22 - protein, 120 aa, Artificial Sequence
  scFv heavy chain variable region
EVQLEESGGGLVKPGGSLKLSCAASGFTFSTYAMSWVRQTPEKRLEWVATISSGGGYTYYPDSVKGRFTISRDNAKNTLYLQMSSLRSEDSAMYYCARIYYYGSRNGYFDHWGQGTTLTV

SEQ ID NO: 23 - protein, 121 aa, Artificial Sequence
  Murine scFv light chain variable region
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKEIKRADAAPTVSS

SEQ ID NO: 24 - DNA, 36 bp, Artificial Sequence
  Heavy Chain Variable Region Forward Primer
ggccagtggatagtcagatgggggtgtcgttttggc

SEQ ID NO: 25 - DNA, 20 bp, Artificial Sequence
  Heavy Chain Variable Region Reverse Primer
gaggtgaagctggtggagtc

SEQ ID NO: 26 - DNA, 21 bp, Artificial Sequence
  Light Chain Variable Region Forward Primer
ggatacagttggtgcagcatc

SEQ ID NO: 27 - DNA, 21 bp, Artificial Sequence
  Light Chain Variable Region Reverse Primer
gatattgtgatgacgcaggct

(19) United States
(12) Patent Application Publication
(10) Pub. No.: US 2023/0052153 A1
(43) Pub. Date: Feb. 16, 2023

(54) TREATING IMMUNE PLATELET DISORDERS USING ANTIGEN-BINDING FRAGMENTS

(71) Applicants: NewSouth Innovations Pty Limited, Sydney, New South Wales (AU); NSW Health Pathology, Westmead, New South Wales (AU); South Eastern Sydney Area Health Service, Kogarah, New South Wales (AU)

(72) Inventors: Beng Hock Chong, Sydney, New South Wales (AU); Halina Leung, Sydney, New South Wales (AU); Jose Perdomo, Sydney, New South Wales (AU)

(21) Appl. No.: 17/442,791

(22) PCT Filed: Mar. 25, 2020
(86) PCT No.: PCT/AU2020/050284

§ 371 Date: Sep. 24, 2021

(30) Foreign Application Priority Data
Mar. 25, 2019 (AU) 2019900991

(51) Int. Cl. C07K 16/24; A61K 47/68; A61P 7/02

(52) U.S. Cl. CPC C07K 16/24; A61K 47/6811; A61P 7/02; A61K 47/6845; C07K 2317/622; C07K 2317/24; C07K 2317/76; A61K 2039/505

(57) ABSTRACT

The present invention relates to compositions and methods for the treatment and prevention of thrombogenic-related diseases and disorders. The compositions may comprise antigen-binding fragments that prevent platelet activation by either blocking Fc_{γ}RIIa binding on platelets, neutrophils and monocytes, or neutralising platelet factor 4.

### TECHNICAL FIELD

The present invention relates to the fields of immunology and medicine. More specifically, the present invention relates to the treatment and prevention of thrombogenic-related diseases and disorders.

### BACKGROUND

Heparin-induced thrombocytopenia (HIT) is a limb- and life-threatening complication of heparin therapy that affects 1-5% of patients receiving unfractionated heparin. HIT and thrombosis are the result of immune reactions to unfractionated (UF) or low molecular weight (LMW) heparin. The reaction is primarily mediated by the HIT antibody, an IgG antibody against heparin-platelet factor 4 (PF4) complex. The antibody-antigen complex binds and cross-links platelet Fc_{γ}RIIa receptors, leading to strong platelet activation and thrombus formation. The immune complex also induces platelet-neutrophil interaction and NETosis. These potent prothrombotic processes (platelet activation, platelet-neutrophil interaction and NETosis) drive thrombosis in HIT. Not surprisingly, the thrombosis in HIT is often extensive and severe, leading to devastating clinical sequelae such as life-threatening arterial thrombosis (e.g. heart attacks and stroke), lung clots (pulmonary embolus), leg gangrene and limb loss, multi-organ failure and death. HIT is relatively common as UF and LMW heparins are widely used in clinical practice. Because of devastating clinical sequalae, it is a drug complication dreaded by many clinicians globally.

The initial step in treating HIT is the withdrawal of heparin. However, this does not stop the strong thrombotic processes and thereby does not prevent serious clinical sequelae such as limb gangrene and thrombotic deaths. Anticoagulant treatments for HIT in particular danaparoid, lepirudin and argatroban are only partially effective in treating thrombosis in HIT. Recently other anticoagulants such as fondaparinux and new oral anticoagulants (e.g. dabigatran) have also been used in an attempt to inhibit blood coagulation events downstream of the Fc_{γ}RIIa-dependent thrombosis-initiating event that drive the chain of potent prothrombotic processes (platelet activation, platelet-leukocyte interaction and NETosis) leading to severe thrombosis. Without extinguishing the initiating/driving events, the use of an anticoagulant alone to inhibit the downstream coagulation pathway events has proven to be inadequate, with these drugs being capable of moderately reducing thrombotic events but failing to significantly reduce the limb gangrene and mortality rates. Furthermore, these drugs are renally excreted and tend to accumulate in the plasma and cause bleeding. Anticoagulant therapy alone does not suppress or extinguish the HIT antibody-induced platelet activation and thrombin generation which together drive thrombosis in HIT.

Immune thrombocytopenias (ITPs) are typically conditions that are mediated by pathogenic IgG antibodies that have specificity for platelet receptors GPIIb-IIIa or GPIb-IX. These antibodies cause peripheral blood platelet destruction leading to a decrease in circulating platelets, thrombocytopenia and bleeding. In some patients these antibodies can activate platelets and cause thrombosis. ITPs can be primarily considered as autoimmune diseases with unknown cause or which are secondary to other autoimmune diseases (e.g. systemic lupus erythematosus), immune reactions to drugs (e.g. drug-induced ITPs) or infection [infection-related ITPs, e.g. ITP associated with human immunodeficiency virus (HIV), Epstein Barr virus (EBV), measles and other infections].

Severe ITPs that are mediated by anti-GPIb-IX IgG antibodies are often very severe and are refractory to treatment with conventional immunosuppressive therapies. The first line treatments include glucocorticosteroids, IVIg, anti-D, or any combination thereof. The second line treatments consist of azathioprine, cyclophosphamide, danazol, vinca alkaloids, rituximab, TPOR-agonists, splenectomy etc. With the exception of TPOR-agonists, anti-GPIb-IX IgG antibody mediated ITP does not seem to respond to conventional immunosuppressive therapies. Even with TPOR-agonists, it typically takes up to 2 weeks for the platelet response to occur, as these drugs act by stimulating megakaryocyte precursors (platelet producing cells) in bone marrow and it takes about 2 weeks for megakaryocyte precursors to mature to a stage that they can produce platelets. During this time if a patient bleeds, there is no effective treatment if the patient has already become refractory to immunosuppressive drugs. Even with TPOR-agonists, 20-30% of patients fail to respond. Third line treatments include combined therapy (first and second line agents), combined chemotherapy and allogenic bone marrow transplantations. The first-, second- and third-line treatments all have serious adverse effects and are not well tolerated. The lack of response of ITP, specifically those mediated by anti-GPIb-IX antibodies is currently unknown but our present studies have shown that the underlying mechanism is probably Fc_{γ}RIIa-dependent.

Current drug therapies have not been effective in controlling thrombosis in HIT and ITPs because they fail to extinguish the potent prothrombotic processes, such as Fc_{γ}RIIa-dependent platelet activation, platelet-neutrophil interaction and NETosis, which drives the thrombosis formation. There is an urgent need for more effective treatments for HIT and ITP.

### SUMMARY OF THE INVENTION

The present invention provides antigen-binding fragments that bind to either the Fc_{γ}RIIa receptor or platelet factor 4 (PF4) to prevent or treat HIT and ITP. The present invention alleviates at least one of the shortcomings of present treatments for HIT and ITP.

According to a first embodiment of the present invention, there is provided an antigen-binding fragment that prevents activation of platelets by either blocking Fc_{γ}RIIa binding on platelets or neutralising PF4.

According to a more specific embodiment of the present invention, there is provided an antigen-binding fragment, optionally an scFv, that specifically binds to Fc_{γ}RIIa, wherein the antigen-binding fragment comprises:
a heavy chain variable region comprising:
   a heavy chain CDR1 sequence according to SEQ ID NO: 3;
   a heavy chain CDR2 sequence according to SEQ ID NO: 4;
   a heavy chain CDR3 sequence according to SEQ ID NO: 5;
wherein said heavy chain variable region comprises at least 90% sequence identity to SEQ ID NO: 13,
and a light chain variable region comprising:
   a light chain CDR1 sequence according to SEQ ID NO: 6;
   a light chain CDR2 sequence according to SEQ ID NO: 7; and
   a light chain CDR3 sequence according to SEQ ID NO: 8;
wherein said light chain variable region comprises at least 90% sequence identity to SEQ ID NO: 14, and
   wherein the heavy chain and light chain variable regions are joined by a flexible-linker region.

According to another specific embodiment of the present invention, there is provided an antigen-binding fragment, optionally an scFv, that specifically binds to PF4, wherein the antigen-binding fragment comprises:
a heavy chain variable region comprising:
   a heavy chain CDR1 sequence according to SEQ ID NO: 16;
   a heavy chain CDR2 sequence according to SEQ ID NO: 17;
   a heavy chain CDR3 sequence according to SEQ ID NO: 18;
wherein said heavy chain variable region comprises at least 90% sequence identity to SEQ ID NO: 22,
and a light chain variable region comprising:
   a light chain CDR1 sequence according to SEQ ID NO: 19;
   a light chain CDR2 sequence according to SEQ ID NO: 20; and
   a light chain CDR3 sequence according to SEQ ID NO: 21;
wherein said light chain variable region comprises at least 90% sequence identity to SEQ ID NO: 23 and
   wherein the heavy chain and light chain variable regions are joined by a flexible-linker region.

According to another embodiment the flexible linker region comprises an oligopeptide sequence of between 10 and 30 amino acids. The oligopeptide may comprise a combination of glycine and serine residues and, according to a specific embodiment, the flexible linker region may comprise an oligopeptide having an amino acid sequence as set forth in SEQ ID NO:9.

According to a specific embodiment the antigen-binding fragment according to the invention comprises an amino acid sequence as set forth in SEQ ID NO: 2 or the amino acid sequence as set forth in SEQ ID NO: 12. According to an embodiment, the antigen-binding fragment according to the invention comprises an amino acid sequence as set forth in SEQ ID NO: 12.

According to another specific embodiment the antigen-binding fragment according to the invention comprises an amino acid sequence as set forth in SEQ ID NO: 15.

According to another embodiment of the present invention, the antigen-binding fragment may be conjugated to an anti-coagulant. In certain embodiments, the anti-coagulant may be selected from the group consisting of Factor Xa, Factor IIa, Factor VIIa/tissue factor, FXII inhibitors, desirudin, tick anticoagulant peptide, factor Xa inhibiting peptides, prothrombin inhibiting peptides, danaparoid, bivalirudin, lepirudin, argatroban and other anticoagulants. In further embodiments the anti-coagulant includes inhibitors that are effective in preventing clot formation, for example, NETosis inhibitors and DNAase. According to specific embodiments, the antigen-binding fragment is conjugated to bivalirudin or lepirudin. According to certain embodiments, the antigen-binding fragment may comprise the amino acid sequence of SEQ ID NO:10 or the amino acid sequence of SEQ ID NO:11, or a variant of either sequence having 1, 2, or 3 amino acid substitutions in the framework region.

According to another embodiment of the present invention, there is provided a nucleic acid molecule encoding an antibody-binding fragment according to the invention. Also provided by the present invention are vectors comprising nucleic acid molecules according to the invention, as well as host cells, optionally mammalian or insect cells, comprising said vectors or nucleic acid molecules

According to another embodiment of the present invention, there is provided a pharmaceutical composition comprising an antigen-binding fragment of the invention.

According to yet another embodiment of the present invention, there is provided a method of treating a subject, optionally a human subject, with a thrombogenic-related disease, optionally ITP or HIT, the method comprising administering to the subject a therapeutically effective amount of at least one antigen-binding fragment of the invention or a pharmaceutical composition of the invention.

According to yet another embodiment of the present invention, there is provided the use of an antigen-binding fragment of the invention for the manufacture of a medicament for the treatment of a thrombogenic-related disease, optionally ITP or HIT.

### BRIEF DESCRIPTION OF THE FIGURES

FIG. 1 shows protein expression of HRU1, HRU2, HRU3 and HRU4. (A) SDS-PAGE coomassie staining (C) and Western blots (W). Molecular weight markers are also shown. Arrows indicate the protein band corresponding to HRU molecules. (B) HRU1, HRU2, HRU3 and HRU4 were incubated with human platelets and washed. Bound scFvs were detected with anti-c-Myc-AF-488. Filled histograms represent negative controls (absence of scFvs) while open histograms represent binding by HRU scFvs. (C) Platelet aggregation was induced by patient IgG P1 to P4 or HIT-like antibody KKO (black bars) and was inhibited by HRU1-3 (left panel) or HRU4 (right panel). (D) Serotonin release was induced by patient IgG P1 to P4 or HIT-like antibody KKO (black bars) and was inhibited by HRU1-3. (left panel) or HRU4 (right panel). H, HRU.

FIG. 2 shows (A) platelet aggregation induced by 0.3 U of thrombin in the absence (black bar) and presence of HRU2 or HRU3. (B) Thrombin Time Delay assay for HRU2 and HRU3. Increasing concentrations of HRU1-3 were incubated with undiluted plasma prepared from ACD anticoagulated blood. The clotting time is shown in seconds.

FIG. 3 shows reconstitution of the HIT condition in a microfluidics chamber. (A) Time course of platelet aggregates on a von Willebrand factor (vWf) coated microfluidics chamber. Thrombus formation images using DiOC₆-labelled whole blood accumulated over the indicated time on a surface coated with vWf. The images show thrombus formation in the present of HIT IgG from a HIT patient plus heparin and inhibition of thrombus deposition in the presence of HRU1-4. (B) The graph shows the percentage area coverage versus time with and without HRU1 for patient sample P4. The IV.3 MoAb was used for comparison. Data are mean±SD (n≥3) (C) The graph shows the percentage area coverage versus time with and without HRU4 for patient samples P1-3 or the HIT-like antibody KKO. Data are mean±SD (n≥3). P, patient; sec or s, seconds; Hep, heparin.

FIG. 4 (A) shows HRU1 binding Tg mouse platelets in vivo. (B-F) Human HIT IgG recapitulates thrombocytopenia in FcγRIIA/hPF4 transgenic mice. Mice were injected with HIT IgG plus heparin (n=4) and platelet numbers determined before treatment (set to 100%) and at the times indicated in the graph. Thrombocytopenia was significantly inhibited by HRU1, HRU2, HRU3 or HRU4 in these animals. *P<0.05; **P<0.01; ****P<0.0001.

FIG. 5—HRU1, HRU2, HRU3 or HRU4 inhibit thrombosis in mice. (A) Mouse platelets were labeled in vivo with anti CD42c-Dylight649. Animals were treated with control IgG or HIT IgG or HIT IgG plus HRU1 (all in the presence of heparin). Lungs collected after treatment were scanned on an IVIS Spectrum CT scanner. The scale bar indicates the level of fluorescence (top panel). Quantitation of lung fluorescence is shown in the graph. Lung sections were stained with hematoxylin and eosin. Thrombi were abundant in HIT IgG treated animals (yellow arrows). Thrombi could not be detected in lung preparations from animals treated with HIT IgG+HRU1 (bottom panel) (B) and (C) HRU4 inhibits thrombosis in mice. Animals were treated with control HIT IgG or KKO with or without HRU4 and heparin. Fixed lungs were scanned on an IVIS Spectrum CT scanner. Green colour in lungs indicates the level of fluorescence. Quantitation of lung fluorescence is shown in the graph. Lung sections were stained with hematoxylin and eosin. Thrombi were abundant in HIT IgG treated animals (yellow arrows). (D and E) Graph showing quantitation of lung fluorescence in lungs of mice treated with HIT IgG with and without HRU2 or HRU3. Thrombi could not be detected in lung preparations from animals treated with HIT IgG+HRUs. *P<0.05; **P<0.01; ***P<0.001; ****P<0.0001.

FIG. 6—HRU1 inhibits thrombocytopenia in anti-GPIX treated mice. (A) Anti-GPIX antibody induced thrombocytopenia in FcγRIIA/hPF4 double transgenic mice (blue line). Thrombocytopenia was potently inhibited by HRU1 (red line). (B) HRU1 inhibits thrombosis in anti-GPIX treated mice. Lung sections were stained with hematoxylin and eosin. Thrombi were absent in animals treated with control IgG but abundant in anti-GPIX antibody treated animals (yellow arrows). Thrombi could not be detected in lung preparations from animals treated with anti-GPIX antibody+HRU1. (C) Mouse platelets were labeled in vivo with anti-CD42c-Dylight649. Animals were treated with control IgG or anti-GPIX or anti-GPIX+HRU1. Platelet rich thrombi (red) were observed in anti-GPIX treated mice but not in HRU1 treated animals. ****P<0.0001

FIG. 7 shows CTBR1 competing with patient HIT IgG for binding to PF4 (ELISA assay using fluorescence labelled proteins). (A) Increasing CTBR1 concentrations reduce binding of HIT IgG to immobilized PF4 (HIT IgGs from two patients, 1&2, are shown). GMF, geometric mean fluorescence. (B) CTBR1 Fab (blue line) inhibits platelet aggregation induced by HIT IgG plus heparin (red line).

FIG. 8 shows synergistic activity of CTBR1 Fab. CTBR1 acts synergistically with suboptimal concentrations of HRU3 and protects animals from (A) HIT IgG-induced thrombocytopenia and (B) HIT IgG-induced thrombosis in mouse lungs. Left panel, white light image of mouse lungs; middle panel, fluorescence (green) represents signal from clots in the lungs; right panel, overlay with fluorescence shown in red. (C) The graph shows quantification of the fluorescence (radiant efficiency) of the lungs shown in B. *P<0.05; **P<0.01; ns, not significant.

### DEFINITIONS

As used in this application, the singular form “a”, “an” and “the” include plural references unless the context clearly dictates otherwise. For example, the term “antigen-binding fragment” also includes multiple antigen-binding fragments.

As used herein, the term “comprising” means “including.” Variations of the word “comprising”, such as “comprise” and “comprises,” have correspondingly varied meanings. Thus, for example, a method of treatment “comprising” administrating an antigen-binding fragment can include one or more additional components (e.g. salts, other immunomodulatory agents, another antigen-binding fragment).

As used herein the term “multiple” means more than one. In certain specific aspects or embodiments, multiple may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or more, and any integer derivable therein, and any range derivable therein.

As used herein, the terms “antibody” and “antibodies” refer to antibodies made up of two heavy chains and two light chains, with both a distinct Fc-region and Fab regions. Antibodies may be of IgG (including IgG1, IgG2, IgG3, and IgG4), IgA (including IgA1 and IgA2), IgD, IgE, IgM, and IgY subclasses.

As used herein, the term “antigen-binding fragment” includes, but is not limited to, Fv, Fab, Fab′ and F(ab′)2, Fd, single-chain Fvs (scFv), single-chain antibodies, disulphide-linked Fvs (sdFv) and fragments comprising either a VL or VH domain. According to an embodiment, antigen-binding fragments according to the invention exclude Fc-regions. In specific embodiments, an antigen-binding fragment refers to an scFv comprising a heavy chain and a light chain domain joined by a flexible linker. The antibody-binding fragments or parts/components thereof may be derived from any animal origin or appropriate production host. Antigen-binding fragments, including single-chain antibodies, may comprise the variable region/s alone or in combination with the entire or partial of the following: hinge region, CH1, CH2, and CH3 domains. Also included is any combination of variable region/s and hinge region/s, CH1, CH2, and CH3 domains. Antibody-binding fragments may be monoclonal, polyclonal, chimeric, humanized, and human monoclonal and polyclonal antibodies which specifically bind the biological molecule.

As used herein, the term “humanized antigen-binding fragment” refers to forms of antigen-binding fragments that contain sequences from human antibodies as well as non-human antibodies (e.g. murine antibodies). For example, a humanized antigen-binding fragment can comprise substantially all of at least one variable domain, in which all/substantially all of the hypervariable loops correspond to those of a non-human immunoglobulin and all/substantially all of the FF region are from the human immunoglobulin sequence.

As used herein, the terms “binding specificity” and “specifically binding” in reference to antibody, antibody variant, antibody derivative, antigen-binding fragment, and the like refers to its capacity to bind a given target molecule preferentially over other non-target molecules. For example, if the antibody, antibody variant, antibody derivative, or antigen-binding fragment (“molecule A”) is capable of “binding specifically” or “specifically binding” to a given target molecule (“molecule B”), molecule A has the capacity to discriminate between molecule B and any other number of potential alternative binding partners. Accordingly, when exposed to multiple different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding molecules that are not molecule B at a weak, yet detectable level. This is commonly known as background binding and is readily discernible from molecule B-specific binding, for example, by use of an appropriate control.

As used herein, the term “therapeutically effective amount”, includes a sufficient, but non-toxic amount of a compound or composition of the invention to provide the desired therapeutic effect. The “therapeutically effective amount” will vary from subject to subject depending on one or more factors amongst for example, the particular agent being administered, the severity of the condition being treated, the species being treated, the age and general condition of the subject, and the mode of administration. For any given case, an appropriate “effective amount” may be determined by one of ordinary skill in the art using routine experimentation. Typically, “therapeutically effective amount” refers to an amount sufficient to result in one or more of the following: recession/reduction in the extent of disease, inhibition of disease growth or progression, cessation of disease growth, relief of disease-imposed discomfort, or prolongation of life of the vertebrate having the disease.

As used herein, the term “subject” includes any animal of economic, social or research importance including bovine, equine, ovine, primate, avian and rodent species. Hence, a “subject” may be a mammal such as, for example, a human or a non-human mammal.

As used herein, the term “treatment”, refers to any and all uses which remedy a disease state or symptoms, or otherwise prevent, hinder, retard, or reverse the progression of disease or other undesirable symptoms in any way whatsoever.

As used herein, the term “pharmaceutically acceptable carrier”, refers to means approved by a regulatory agency of the Federal or State government or other generally recognized pharmacopeia for use in animals, and more particularly humans. The term ‘carrier’ refers to a diluent, adjuvant (e.g. complete or incomplete Freund's adjuvant), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin. Such compositions will contain a prophylactically or therapeutically effective amount of carrier so as to provide the form for the proper administration to the subject. The formulation should suit the mode of administration. In a preferred embodiment, the pharmaceutical compositions are sterile and in suitable form for administration to a subject, preferably a mammalian subject, and more preferably a human subject.

As used herein, the term “isolated” in reference to a biological molecule (e.g. an antigen-binding fragment) is a biological molecule that is free from at least some of the components with which it naturally occurs.

As used herein, the terms “protein” and “polypeptide” each refer to a polymer made up of amino acids linked together by peptide bonds and are used interchangeably. For the purposes of the present invention a “polypeptide” may constitute a full-length protein or a portion of a full length protein.

As used herein, the term “Glycoprotein (GP) Ib/IX complex” refers to a multiunit complex consisting of GPIb-alpha, GPIb-beta, GPIX and GPV. The complex is abundantly present on platelets and is required for platelet adhesion. Autoantibodies developed in immune platelet disorder target components of the GPIb/IX complex and cause thrombocytopenia and thrombosis.

The term ‘Fc_{γ}RIIa’ refers to an Fc receptor protein found on the surface of certain cells, in particular human platelets, macrophages and neutrophils, that contribute to the protective functions of the immune system. Fc receptors have binding specificity for a part of an antibody known as the Fc (Fragment, crystallizable) region. The CD32 immunoglobulin G (IgG) receptor (Fc_{γ}RIIa) may be a human Fc_{γ}RIIa receptor (e.g. as defined by a sequence set forth in any one of: NCBI reference sequence accession no. NP_067674.2 or NP_001129691.1). In some embodiments the Fc_{γ}RIIa receptor protein may not include a signal peptide and/or a propeptide. Additionally, or alternatively, the anti-Fc_{γ}RIIa receptor antigen-binding fragment may be capable of binding specifically to antigenic epitopes present in a Fc_{γ}RIIa receptor variant (e.g. Fc_{γ}RIIa isoform, splice variant or allotype). For example, the anti-Fc_{γ}RIIa receptor antigen-binding fragment can bind an epitope within the D1 domain or the D2 domain or an epitope that is formed when association occurs between the two domains. The CD32 immunoglobulin G (IgG) receptor (Fc_{γ}RIIa) is a potential therapeutic target for diseases in which IgG immune complexes (ICs) mediate inflammation.

The term ‘PF4’ refers to platelet factor 4 a small heparin-binding protein secreted by certain cells, in particular activated platelets. The biological activity of PF4 is unclear. However, by itself PF4 may bind and neutralize the anticoagulant activity of heparin and heparin-like negatively charged molecules in blood. The PF4 may be human PF4 (e.g. as defined by a sequence set forth in any one of: NP_001350281.1 or NP_002610.1). In some embodiments the PF4 protein may not include a signal peptide and/or a propeptide. Additionally, or alternatively, the anti-PF4 antigen-binding fragment may be capable of binding specifically to antigenic epitopes present in a PF4 protein variant (e.g. PF4 isoform, splice variant or allotype). For example, the anti-PF4 antigen-binding fragment may bind an epitope within the heparin-binding domain or outside this domain, or an epitope that forms when PF4 interacts with its carrier proteoglycan carrier protein.

As used herein, the term “scFv” refers to a single chain variable fragment generated by joining a single variable heavy chain and light chain domains of an antibody. The scFv retains the binding specificity of the parental antibody.

As used herein, the term “kit” refers to any delivery system for delivering materials. Such delivery system includes systems that allow for the storage, transport, or delivery of reaction reagents (e.g. labels, reference samples, supporting materials, etc. in the appropriate containers) and/or supporting materials (e.g. buffers, written instructions for administering the agent etc.) from one location to another. For example, kits may include one or more enclosures, such as boxes, containing the relevant reaction reagents and/or supporting materials. The term “kit” includes both fragmented and combined kits. A “fragmented kit” refers to the delivery system comprising two or more separate containers that each contain a sub-portion of the total kit components. The containers may be delivered to the intended recipient together or separately. Any delivery system comprising two or more separate containers that each contain a sub portion of the total kit components are included within the meaning of the term “fragmented kit”. A “combined kit” refers to a delivery system containing all the components for delivery in a single container (e.g. a single box housing each of the desired components).

As used herein, the term “about” when used in reference to a recited numerical value includes the recited numerical value and numerical values within plus or minus ten percent of the recited value.

As used herein, the term “between” when used in reference to a range of numerical values encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### DETAILED DESCRIPTION

A need continues to exist for more effective treatments of HIT and thrombosis associated with ITP or an immune platelet disorder, in which the specific receptors are targeted to inhibit antibody-induced platelet activation and neutrophil activation via Fc_{γ}RIIa, providing a more effective treatment and/or reducing side effects.

Platelet activation occurs after vascular injury, preventing blood loss and is essential to maintenance of body homeostasis. However, platelet activation is also part of pathogenic processes such as thrombus formation in thrombogenic-related diseases and disorders. This is encompassed by immune platelet disorders which include immune thrombocytopenia, HIT or ITP. HIT is caused by heparin administration resulting in the activation of platelets via an immune-mediated reaction comprising autoantibody reacting with blood factor called platelet factor 4 (PF4) in the presence of heparin. This complex then activates platelets via association with the Fc_{γ}RIIa receptor. Platelet activation then leads to clot formation that can result in the need for limb amputation, or in more serious cases, death. Whilst the use of anticoagulants is the current accepted practise, these have limited effect and often have severe side effects. For example, lepirudin is a recombinant peptide that is a direct thrombin-inhibitor, but when administered it acts dose-dependently with a short half-life due to proteolysis. As such, dose control is difficult with bleeding occurring as a result of over-dosage. Furthermore, the withdrawal of heparin during HIT does not produce rapid improvements, as the HIT antibody continues to act via interaction with PF4 complexed with endogenous polyanions such as chondroitin sulphate or nucleic acids.

Similarly, platelet disorders, in particular ITP, include a heterogenous group of disorders that are principally caused by autoantibodies that recognize glycoproteins present on the surface of platelets and megakaryocytes (platelet precursor cells). Autoantibodies recognize abundant platelet glycoproteins, mainly the fibrogen receptor, GPIIb/IIIa or the von Willebrand factor receptor, GPIb/IX. These antibodies cause a reduction in the total platelet counts by several mechanisms including platelet destruction in the spleen, decreased platelet production by megakaryocytes and platelet uptake by the liver.

The platelet disorders encompassed within “ITP”, caused by autoantibodies against the GPIb/IX complex, elicit particular pathogenic actions that cause severe thrombocytopenia and thrombosis and are less responsive to some therapeutic interventions such as intravenous immunoglobulin treatment. Antibodies against GPIb/IX may cause platelet desialylation and subsequent platelet clearance by the liver. A perhaps paradoxical aspect of ITP is the propensity of patients to develop thrombosis even when the platelet count is low. The mechanism that accounts for this observation is unclear but suggests that it may be related to platelet activation induced by certain autoantibodies. This hypothesis is supported by findings, using transgenic mice expressing the human Fc_{γ}RIIa receptor (Fc_{γ}RIIa or CD32) on their platelets, that an antibody against the GPIX subunit of the GPIb/IX complex caused both thrombocytopenia and thrombosis. Antibodies against GPIb/IX, which are found in ITP, can lead to both platelet destruction and platelet activation and thrombosis. Pathogenic aspects of other immune conditions such as drug-induced thrombocytopenia (DITP), systemic lupus erythematosus (SLE) and rheumatoid arthritis also depend on activation of and/or signalling by the Fc_{γ}RIIa receptor.

Without wishing to be bound by theory it is postulated that the present invention works by antigen-binding fragments blocking the Fc_{γ}RIIa receptor or blocking PF4 binding to its respective ligand and thereby preventing thrombus formation.

Accordingly, certain embodiments of the present invention provide Fc_{γ}RIIa-specific and PF4-specific antigen-binding fragments and pharmaceutical compositions comprising antigen-binding fragments and variants thereof. Other embodiments of the present invention relate to methods of treating thrombogenic-related diseases in subjects afflicted with the same. Further aspects of the present invention relate to medicaments comprising antigen-binding fragments, and methods for their preparation. Also contemplated by the present invention are small molecule inhibitors of Fc_{γ}RIIa or PF4 that bind to the receptor and inhibit its function.

### Fc_{γ}RIIa-Specific and PF4-Specific Antigen-Binding Fragments

The present invention embodies methods, pharmaceutical compositions and medicaments comprising at least one Fc_{γ}RIIa-specific and/or PF4-specific antigen-binding fragment, derivative or variant thereof.

An anti-Fc_{γ}RIIa or PF4-specific antigen-binding fragment according to the invention may comprise a heavy chain and a light chain which may or may not be derived from the same source.

The antigen-binding fragments and/or variants thereof according to the present invention are not restricted to any particular isotype. In some embodiments, they are humanized antigen-binding fragments and/or variants thereof.

Included within the scope of the present invention are “fragments” of antibodies. In general, the fragments are “antigen binding fragments” in the sense that they are capable of specifically binding to an antigen/epitope (e.g. Fc_{γ}RIIa or PF4) as the parent antibody from which they are derived or upon which they are based. Typically, an antigen binding fragment retains at least 10% of the antigen/epitope binding capacity of the parent antibody, or, at least 25%, 50%, 60%, 70%, 80%, 90%, 95%, 99% or 100% of the antigen/epitope binding capacity of the parent antibody. It is also contemplated that an antigen binding fragment of an antibody described herein may include conservative amino acid substitutions that do not substantially alter its antigen/epitope binding specificity/capacity (e.g. at least 70%, 80%, 90%, 95%, 99% or 100%, of its antigen/epitope binding specificity/capacity (e.g. at least 70%, 80%, 90%, 95%, 99% or 100% of its antigen/epitope binding specificity/capacity may be retained).

Non-limiting examples of antigen-binding fragments include portions of a full-length antibody, peptide and derivatives thereof including, for example Fab, Fab′, F(ab)₂, F(ab)₃, Fv, single-chain Fc (scFv), dsFv, Fd fragments, Fab fragments molecules (e.g. scFv), minibodies, diabodies, triabodies, tetrabodies, kappa bodies, linear antibodies, multispecific antibody fragments formed from antibody fragments, and any portion or peptide sequence of the antibody that is capable of specifically binding to the relevant antigen/epitope (e.g. Fc_{γ}RIIa or PF4). A further non-limiting example includes a single-chain antigen-binding fragment comprising a heavy chain variable region and a light chain variable region connected by a linker, wherein the antigen-binding fragment regions may be homologous or heterologous. An even further non-limiting example includes a single-chain antigen-fragment comprising a heavy chain variable region and a light chain variable region linked by a flexible linker.

Specific examples of antigen-binding fragments specific for Fc_{γ}RIIa according to the present invention include the scFvs HRU1 to HRU4. An example of an antigen-binding fragment specific for PF4 according to the present invention is CTBR1.

HRU1 (comprising an amino acid sequence as set forth in SEQ ID NO: 2) is an scFv derived from mouse IV.3 monoclonal antibody (mAb) constructed by joining single variable heavy chain and light chain domains of the IV.3 antibody with a flexible linker and has undergone some mutation framework modifications to humanize the scFv. HRU2 and HRU3 comprise HRU1 conjugated to bivalirudin (HRU2; comprising an amino acid sequence as set forth in SEQ ID NO: 10) or lepirudin (HRU3; comprising an amino acid sequence as set forth in SEQ ID NO: 11). HRU4 (comprising an amino acid sequence as set forth in SEQ ID NO: 12) is also a scFv constructed by joining single variable heavy chain and light chain domains of the IV.3 antibody with a flexible linker, and which has undergone further mutational framework modifications to obtain the best function—hence a single-chain humanized antibody fragment “scFv”. HRU4 can be conjugated with anticoagulant peptides (e.g. bivalirudin and lepirudin which are often used in HIT treatment) to create a bifunctional agent, i.e. an anti-thrombotic and anticoagulant agent.

HRU1 and HRU2 both comprise the same light and heavy chain variable region CDRs, with heavy chain variable region CDRs of SEQ ID NOs 3, 4 and 5, and light chain variable region CDRs of SEQ ID Nos: 6, 7 and 8, and a Fc_{γ}RIIa binding fragment according to the present invention will comprise those CDRs, whereas some modification of the framework region/s, including one or more conservative or humanization substitutions or modifications is contemplated, as described below.

CTBR1 (comprising an amino acid sequence as set forth in SEQ ID NO: 15) is an scFv derived from a murine monoclonal antibody specific for human PF4, constructed by joining single variable heavy chain and light chain domains of said antibody with a flexible linker. CTBR1 comprises the heavy chain variable region CDRs of SEQ ID NOs: 16, 17 and 18, and the light chain variable region CDRs of SEQ ID NOs 19, 20 and 21, and a PF4 binding fragment according to the present invention will comprise those CDRs whereas, again, some modification of the framework region/s, including one or more conservative substitutions or modifications is contemplated, as described below.

The linker used to connect the variable region of heavy and light chains may be any suitable linker for use in protein chemistry as known in the art. The linker may be substantially linear in nature, or may be branched, but allows for the heavy peptide portion of the antigen-binding fragment to move independently of the light chain portion thereof, and may be, for example, a linear C1-050 linker comprising any suitable organic moiety/ies. In certain embodiments, the linker may comprise an oligopeptide, or may be peptidomimetic (comprising a number of, or only amino acid analogues). The linker may be derived from a native sequence, a variant thereof, or a synthetic sequence. Oligopeptide or peptidomimetic linkers may comprise, e.g. naturally occurring, non-naturally occurring amino acids, or a combination of both. A non-limiting example of a linker for use in the antigen-binding fragments according to the present invention may comprise an oligopeptide of between 5-60 amino acids, which may, for example, comprise regular series of glycine and 1 to 3 serines to enhance solubility. According to an embodiment, the linker for joining the heavy chain and light chain portions of scFvs according to the invention is a 17-amino acid serinesglycine₁₂ linker having an amino acid sequence as set forth in SEQ ID NO: 9.

Also included within the scope of the invention are “derivatives” of antigen-binding fragments described herein. A “derivative” of an antigen-binding fragment of the present invention refers to an antigen-binding fragment described herein that is modified to incorporate additional components or have existing component/s altered, but is still capable of specifically binding to the same antigen/epitope (e.g. Fc_{γ}RIIa or PF4) as the parent antibody from which it is derived. Typically, an antigen-binding fragment derivative as contemplated herein retains at least 10% of the antigen/epitope binding capacity of the parent antibody, or, at least 25%, 50%, 60%, 70%, 80%, 90%, 95%, 99% or 100% of the antigen/epitope binding capacity of the parent antibody from which it derived.

Non-limiting examples of modifications suitable to form antibody derivatives include amidation, glycosylation, phosphorylation, peglylation, lipidation, linage to a cellular ligand or other protein, derivatization by known protecting/blocking groups, acetylation, and the like. Additionally or alternatively, the derivative may contain one or more non-classical amino acids.

The antigen-binding fragment derivatives may be formed from covalent modification of the antigen-binding fragment described herein, for example, by reacting targeted amino acid residues of the antigen-binding fragment with an agent capable of reacting with selected side chains or terminal residues. For example, derivatization with bifunctional agents is a useful means for cross-linking an antigen-binding fragments to macromolecular carriers such as water-insoluble support matrices. Antigen-binding fragment and derivatives thereof as contemplated herein may have an agent attached to a base of the antigen-binding fragment capable of increasing its half-life in vivo (e.g. extending the length of time before clearance from the blood stream). A non-limiting example of such a technique includes addition of PEG moieties.

In certain embodiments, the antigen-binding fragment derivative may be a multimer, such as, for example a dimer, comprising one or more monomers, where each monomer includes (i) an antigen binding region as described herein, or a polypeptide region derived therefrom (such as, for example, by conservative substitution of one or more amino acid/s), and (ii) a multimerizing (e.g. dimerising) polypeptide region, such that the antigen-binding fragment forms multimers (e.g. homodimers) that specifically bind to antigens/epitopes (e.g. Fc_{γ}RIIa or PF4). For example, an antigen-binding region of anti-Fc_{γ}RIIa as described herein or polypeptide region derived therefrom, may be recombinantly or chemically fused with a heterologous protein, wherein the heterologous protein comprises a dimerization or multimerization domain. The derivative may be subjected to conditions allowing formation of a homodimer or heterodimer. The heterodimer may comprise identical dimerization domains but different anti-Fc_{γ}RIIa antigen-binding fragments, identical anti-Fc_{γ}RIIa antigen-binding fragments but different dimerization domains, or different anti-Fc_{γ}RIIa antigen-binding fragments and different dimerization domains. Suitable dimerization domains include those that originate from transcription factors (e.g. basic region leucine zipper and zinc fingers), a basic-region helix-loop-helix protein, and an immunoglobulin constant region (e.g. heavy chain constant region or domain thereof such as a CH1 domain, a CH2 domain, or a CH3 domain).

Included within the scope of the present invention are “variants” of the antigen-binding fragments described herein. A “variant” antigen-binding fragment refers to an antigen-binding fragment which alters the amino acid sequence from a “parent” antigen-binding fragment amino acid sequence by virtue of addition, deletion, and/or substitution of one or more amino acid residue/s in the parent antigen-binding fragment sequence. For example, the variant antigen-binding fragment may comprise one or more amino acid substitution/s in one or more CDR and/or framework region/s of the parent antigen-binding fragment (e.g. between 1 and 10, between 2 and 5, or 1, 2, 3, 4 or 5 substitutions in one or more heavy and/or light chain CDR and/or framework regions of the parent antigen-binding fragment). The antigen-binding fragment variant may comprise a heavy chain variable domain sequence and/or a light chain variable domain sequence amino acid sequence having at least 50%, at least 60%, at least 70%, at least 80%, at least 85%, at least 90%, at least 95%, or at least 98% amino acid sequence homology (i.e. sequence identity) with the corresponding variable domain of the parent antigen-binding fragment.

Sequence homology or identity between two sequences is defined herein as the percentage of amino acid residues in the candidate sequence that are identical with the parent antigen-binding fragment residues, after aligning the sequences and introducing gaps, if necessary, to achieve the maximum percent sequence identity. If the two sequences which are to be compared with each other differ in length, sequence identity relates to the percentage of amino acid residues of the shorter sequence which are identical with the amino acid residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive, Madison, Wis., 53711) and/or the program “fasta20u66” (version 2.0u66, September 1998 by William R. Pearson and the University of Virginia; see also W. R. Pearson (1990), Methods in Enzymology 183, 63-98).

In some embodiments, a variant antigen-binding fragment as described herein may differ from a parent antigen-binding fragment by way of conservative amino acid change/s in the sequence of variable antigen-binding fragment. A “conservative change” refers to an alteration that is substantially antigenically or conformationally neutral, producing minimal changes in the tertiary structure of the variant antigen-binding fragment, or producing minimal changes in the antigenic determinants of the variant antigen-binding fragment, as compared the parent antigen-binding fragment, and one which does not render the derivative incapable of binding to the same epitope in the respective antigen as the parent antigen-binding fragment. Non-limiting examples of conservative amino acid changes include substitution of hydrophobic amino acids and substitutions of physiochemically similar amino acids. Persons of ordinary skill in the art can routinely and without difficulty assess whether a given amino acid substitution can be made while maintaining conformational and antigenic neutrality (see, for example Berzofsky, (1985) Science 229:932-940; Bowie et al. (1990) Science 247: 1306:1310)

Alterations in protein conformation may be achieved using well-known assays including, but not limited to, microcomplement fixation methods (see Wasserman et al. (1961) J. Immunol. 87:290-295; Levine et al. (1967) Meth. Enzymol. 11:928-936) and through binding studies using conformation-dependent monoclonal antibodies (see Lewis et al. (1983) Biochem. 22:948-952. The conservative amino acid change/s may occur in one or more CDR and/or framework region/s of the parent antigen-binding fragment (e.g. between 1 and 10, between 2 and 5, or 1, 2, 3, 4, or 5 conservative substitutions in one or more CDR and/or framework regions of the parent antigen-binding fragment).

The antigen-binding fragment of the present invention may comprise a humanized derivative of a non-human antigen-binding fragment as described herein. A “humanized” antigen-binding fragment as contemplated herein is a human/non-human chimeric antigen-binding fragment that contains a minimal sequence derived from non-human immunoglobulin. For example, a humanized antigen-binding fragment may be a human immunoglobulin (recipient antibody) in which residues from CDR region/s of the recipient are replaced by residues from a CDR region of a non-human species (donor CDR) (e.g. a mouse, rat, rabbit, or non-human primate having some desired specificity and affinity for an Fc_{γ}RIIa or PF4). Framework region (FR) residues of the human immunoglobulin may also (optionally) be replaced by corresponding non-human residues, and in some cases humanized antigen-binding fragment may comprise residues not present in the recipient antigen-binding fragment or in the donor antigen-binding fragment to enhance the antigen-binding fragment's performance.

Further contemplated herein are “chimeric” antigen-binding fragment derivatives in which a portion of the heavy and/or light chain is identical with or homologous to corresponding sequences of an antigen-binding fragment described herein derived from a particular species or belonging to a particular antibody class or subclass, while the remainder of the chain/s is/are identical with or homologous to corresponding sequences in antigen-binding fragment derived from another different species or belonging to another different antibody class or subclass. For example, a chimeric antigen-binding fragment as contemplated herein may comprise variable regions heavy chain region derived from an Fc_{γ}RIIa or PF4 antigen-binding fragment as described herein, and light chain region derived from a second species. Chimeric antigen-binding fragments may be generated, for example, by genetic engineering of immunoglobulin gene segments belonging to different species.

In general humanized, chimeric, derivative, antigen-binding fragments as contemplated herein are still capable of specifically binding to the same antigen/epitope (e.g. anti-Fc_{γ}RIIa or anti-PF4) as the parent antibody or antigen-binding fragment from which they derive or which they contain component(s). Typically, they may retain at least 10% of the antigen-epitope binding capacity of the parent antibody or antigen-binding fragment, or at least 25%, 50%, 60%, 70%. 80%, 90%, 95%, 99% or 100% of the antigen/epitope binding capacity of the parent antibody or antigen-binding fragment. For example, they may have a stronger binding affinity and/or binding specificity compared to the parent antibody or antigen-binding fragment.

The capacity of an antigen-binding fragment, derivative, or variant to bind specifically to an antigen/epitope that is targeted by the parent antibody or antigen-binding fragment (i.e. Fc_{γ}RIIa or PF4 antigen/epitope) can be tested using known methods in the art including, for example, competitive and non-competitive assay systems using techniques such as Western blots, radioimmunoassay, enzyme linked immunosorbent assay (ELIS A), immunoprecipitation assays, “sandwich” immunoassays, immunodiffusion assays, precipitin reactions, protein A immunoassays, fluorescent immunoassays, gel diffusion precipitin reactions, complement fixation assays, immunoradiometric assays, agglutinination assays and the like (see, for example, Ausubel et al., eds., Short Protocols in Molecular Biology (John Wiley & Sons, Inc., New York, 4^{th} ed. 1999); Harlow & Lane, Using Antibodies: Laboratory Manual (Cold Spring Harbor Laboratory Press, Cold Spring Harbor, N.Y., 1999).

### Immunoconjugate Agents

The antigen-binding fragments according to the present invention may be conjugated to at least one anticoagulant agent to assist in the treatment or prevention of a thrombogenic-related disease. The anticoagulant may be any inhibitor or compound that inhibits the coagulation pathway such as Factor Xa inhibitors, prothrombin (Factor IIa) inhibitors, Factor VIIa/tissue factor inhibitors, FXII inhibitors, desirudin, tick anticoagulant peptide, inhibitors, danaparoid, bivalirudin, lepirudin, argatroban and other anticoagulants. In further embodiments the anticoagulant include inhibitors that are effective in preventing clot formation, for example, NETosis inhibitors and DNase. According to certain embodiments, an antigen-binding fragment according to the invention may be conjugated to bivalirudin or lepirudin, and may, for example, comprise the amino acid sequence as set forth in SEQ ID NO: 10 or SEQ ID NO: 11. The antigen-binding fragment may be joined to the anticoagulant via a variety of linkers. According to an embodiment the linker is a Ser-Gly linker comprising a plurality of glycine and/or serine residues.

The antigen-binding fragment derivatives may also include labelled antigen-binding fragments such as, for example, antigen-binding fragments labelled with radioactive iodine, indium, sulphur, carbon, tritium, or the like; antigen-binding fragments conjugated with avidin or biotin, antigen-binding fragments conjugated with enzymes (e.g. horseradish, glucose 6-phosphate dehydrogenase glucose oxidase, beta-D-galactosidase, alkaline phosphatase, glocoamylase, acetylcholine esterase, carboxylic acid anhydrase, malate dehydrogenase, lysozyme, or peroxidase), and antigen-binding fragments conjugated with chemiluminescent agents (e.g. acridine esters), bioluminescent agents (e.g. luciferase), or fluorescent agents (e.g. phycobiliproteins).

### Conjugated Antigen-Binding Fragment Production

Processes for the preparation of antigen-binding fragments, derivatives and variants thereof, are well known, and if necessary such known methods may be readily modified, tweaked or adapted without difficulty by persons of ordinary skill in the art in the field of immunology.

### Medicaments and Pharmaceutical Formulations

Medicaments and pharmaceutical formulations according to the present invention comprise antibody-binding fragments, as described herein. The medicaments and pharmaceutical formulations may be prepared using methods known to those ordinary skill in the art. Non-limiting examples of suitable methods are described in Gennaro et al. (Eds), (1990), “Remington's Pharmaceutical Sciences”, Mack Publishing Co., Easton, Pa., USA.

The medicaments and pharmaceutical formulations may comprise one or more pharmaceutically acceptable carriers, excipients, diluents and/or adjuvants which do not produce adverse reaction(s) when administered to a particular subject such as human or non-human animal. Pharmaceutically acceptable carriers, excipients, diluents and adjuvants are generally also compatible with other ingredients of the medicaments and pharmaceutical formulations. Non-limiting examples of suitable excipients, diluents, and carriers can be found in the “Handbook of Pharmaceutical Excipients” 4^{th} Edition, (2003) Rowe et al. (Eds), The Pharmaceutical Press, London, American Pharmaceutical Association, Washington, and may include, for example, distilled water, saline solution, vegetable based oils such as peanut oils, safflower oil, olive oil, cottonseed oil, maize oil, sesame oil, arachis oil, or coconut oil, silicon oil, including polysiloxanes, volatile silicones, minerals oils such as lipid paraffin, soft paraffin or squalene, cellulose derivates such as methyl cellulose, ethyl cellulose, carboxymethylcellulose, sodium carboxymethylcellulose or hydroxypropylmethylcellulose, lower alkanols (for example ethanol or isopropoanol), lower aralkanols, lower polyalkylene glycols or lower alkylene glycols (for example polyethylene glycol, polypropylene glycol, ethylene glycol, propylene glycol, 1,3-butylene glycol), or glycerin, fatty acid esters such as isopropyl palmitate, isopropyl myristate or ethyl oleate, polyvinylpyrridone, agar, carrageenan, gum tragacanth or gum acacia and petroleum jelly. Typically, the carrier or carries will form from 10% to 99.9% by weight of the compositions.

In certain embodiments, medicaments and pharmaceutical formulations of the present invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, intradermal, subcutaneous, intramuscular or intravenous injection.

Solid forms of the medicaments and pharmaceutical formulations for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monosterate or glyceryl distearate.

Liquid forms of the medicament and pharmaceutical formulations for oral administration may contain, in addition to the above agents, a liquid carrier. Suitable liquid carriers include water, oils, such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administrations may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

For preparation of the medicaments and pharmaceutical formulations as injectable solutions or suspensions, non-toxic parenterally acceptable diluents or carriers may be used such as Ringer's solution, isotonic saline, phosphate buffered saline, ethanol and 1,2 propylene glycol.

Emulsions for oral administration may further comprise one or more emulsifying agents. Suitable emulsifying agents include dispersing agents as exemplified above or natural gums such as guar gum, gum acacia or gum tragacanth.

Topical formulations comprise an active ingredient(s) (e.g. i.e. antibodies and/or antigen-binding fragments thereof of the present invention) together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

When formulated as drops, the medicaments and pharmaceutical formulations may comprise sterile aqueous or oily solutions or suspensions. These may be prepared by dissolving the active ingredient in an aqueous solution of bactericidal and/or fungicidal agent and/or any other suitable preservative, and optionally including a surface active agent. The resulting solution may then be clarified by filtration, transferred to a suitable container and sterilised. For example, sterilisation may be achieved by filtration followed by transfer to a container by aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

When formulated as creams, ointments or pastes, the medicaments and pharmaceutical formulations may be semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap, a mucilage, an oil of natural origin such as almond, corn, arachis, castor or olive oil, wool fat or its derivatives, or a fatty acid such as stearic or oleic acid together with an alcohol such as propylene glycol or macrogols.

The medicaments and pharmaceutical formulations may include any suitable surfactant such as an anionic, cationic or non-ionic surfactant such as sorbitan esters or polyoxyethylene derivatives thereof. Suspending agents such as natural gums, cellulose derivatives or inorganic materials such as colloidal silicas, and other ingredients such as lanolin, may also be used.

### Methods for Treating Thrombogenic-Related Diseases

HIT is a life threatening thrombotic complication of heparin treatment. HIT occurs when immune complexes consisting of PF4 and specific HIT immunoglobulins (IgG) are formed following sensitization of the patients by heparin. These immune complexes interact with platelets via Fc_{γ}RIIa receptors, inducing receptor cross-linking, thus leading to platelet activation.

The activated platelets subsequently release PF4 which amplifies the immune complex-mediated platelet activation events and also generates procoagulant microparticles which initiate the downstream coagulation pathway. The clinical consequences include venous thrombosis such as deep vein thrombosis and pulmonary embolism; arterial thrombosis such as myocardial infarction, stroke and limb gangrene which often requires amputation. The antigen-binding fragments and treatment methods herein provide an ability to block the detrimental activity of HIT antibody/PF4/polyanion immune complexes.

Pathogenic aspects of other immune conditions such as immune thrombocytopenia (ITP) an autoimmune bleeding disorder characterised by production of auto-reactive antibodies, drug-induced thrombocytopenia (DITP), systemic lupus erythematosus (SLE) and rheumatoid arthritis also depend on activation and/or signalling via the Fc_{γ}RIIa receptor. Effective inhibition by anti-Fc_{γ}RIIa antigen-binding fragments described herein could additionally alleviate these serious diseases.

ITP can be primary or secondary to other conditions. Primary ITP is defined as an isolated platelet count of less than 100×10⁹/L (reference count 150-400×10⁹/L) in the absence of other causes or conditions that may cause thrombocytopenia. Secondary ITP is due to many conditions including a number of drugs (rifampicin, vancomycin, quinine), H. pylori infection, HIV, hepatitis C, lupus, other automimmune disorders, anti-phospholipid syndrome, and malignancy.

The identification of subjects with or at risk of developing thrombogenic-related diseases, in particular HIT and ITP, is well known to those of ordinary skill in the art. For example, in the case of HIT, the criteria for diagnosis is usually a normal platelet count before the initiation of heparin, thrombocytopenia defined as a drop in platelet count by 30% to <100×10⁹/l or a drop of >50% from the subject's baseline platelet count, the onset of thrombocytopenia typically 5-10 days after initiation of heparin treatment, which can occur earlier with previous exposure to heparin (within 100 days), haematology, acute thrombotic event and HIT antibody seroconversion.

For example, primary ITP is usually defined by low platelet count, normal bone marrow and the absence of other causes of thrombocytopenia. ITP can be diagnosed using standard tests including: urinalysis, CBC with differential, haematology, coagulation, serum chemistry, surfactant D, erythrocyte sedimentation rate, and C-reactive proteins. HIT and ITP (primary or secondary) may develop bleeding, thrombosis and end-organ damage.

Once a subject has been determined as suffering or being at risk of developing a thrombogenic-related disease, such as HIT and ITP, the methods of the present invention may be used for treating those diseases in the subject.

It will be understood that “treating” within the context of the methods described herein refers to the alleviation, in whole or in part of symptoms associated with either ITP or HIT, or slowing, inhibiting or halting further progression of either ITP or HIT. Typically, a therapeutic response can be considered as an increase in platelet count above those thresholds that place the subject at risk for bleeding (30×10⁹), improvement/resolution of thrombosis damage and end-organ damage.

A non-limiting example of the present invention includes administration of antigen-binding fragment, to a subject with moderate to severe ITP (primary or secondary) wherein the subject has either bleeding, thrombosis, deep vein thrombosis (DVT), petechiae or bruising. A further non-limiting example of the present invention includes administration of antigen-binding fragment, to a subject with moderate to severe HIT wherein the subject has either had a reduced or inadequate response to warfarin or other anticoagulants.

A non-limiting example of the present invention includes administration of antigen-binding fragment, to a subject with other immune conditions with thrombocytopenia, and/or thrombosis, and/or end-organ damage. Further non-limiting examples include, NETs-induced thrombo-embolism, organ-injury, other NETs-associated disorders, ITP associated with drugs, viral infections or antibody treatments, antiphospholipid syndrome, cancer-induced thrombocytopenia and thrombo-embolism, autoimmune or inflammatory diseases involving Fc_{γ}RII (CD32) and involving either one or more of the following cells; platelets, neutrophils, monocytes, macrophages, eosinophils, basophils and mast cells.

In a particular embodiment of the invention, the method comprises a combination of one or more antibody-binding fragments, wherein the combination further comprises administering to the subject, two, three, four, five, six, or more antibody-binding fragments described herein. In a further embodiment, the method comprises administering to a subject two or more treatments that act together to inhibit Fc_{γ}RIIa and/or PF4 antibody-binding.

In some embodiments the antigen-binding fragment(s) are administered to a subject in an amount and time that is sufficient to generate an improvement, preferably a sustained improvement, in at least one indicator of disease severity that can be treated. Various indicators may be used to assess whether the amount and time of treatment is sufficient. Such indicators include, for example, clinically recognised indicators of disease severity, symptoms and/or manifestations of the disorder or condition. The degree of improvement is generally determined by a physician, who may make the determination using signs, symptoms, biopsies or other test results.

The subject may be any animal that can benefit from the administration of antigen-binding fragment. In some embodiments, the subject is a mammal, for example, a human, a dog, a cat, a horse, a cow, a pig, a primate, or rodent (e.g. a mouse or rat).

The methods may involve the administration of a “therapeutically effective amount” of antigen-binding fragment according to the invention. A “therapeutically effective amount” will be understood to refer to an amount of antigen-binding fragment that alleviates, in whole or in part symptoms associated with either HIT or ITP, or slows, inhibits or halts further progression or worsening of those symptoms, in a subject with or at risk of developing either HIT or ITP.

The therapeutically effective amount may vary depending upon the route of administration, the particular antigen-binding fragment and the dosage form. Effective amounts of antigen-binding fragments, according to the present invention typically fall in the range of about 0.001 up to 100 mg/kg/day, for example in the range of about 0.05 up to 20 mg/kg/day. Typically, the antigen-binding fragments provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Generally, an effective dosage of antigen-binding fragment, according to the present invention is expected to be in the range of about 0.0001 mg to about 1000 mg of active component(s) (i.e. of antigen-binding fragment according to the present invention) per kg of body weight per 24 hours; typically, about 0.001 mg to about 750 mg per kg body weight per 24 hours; about 0.01 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m² of active component(s) (i.e. antigen-binding fragments according to the present invention). Generally, an effective dosage is expected to be in the range of about 0.1 to about 500 mg/m², preferably about 1 to about 250 mg/m², more preferably about 1 to about 200 mg/m², preferably about 1 to about 150 mg/m², more preferably about 1 to about 100 mg/m², still more preferably about 1 to about 50 mg/m², even more preferably about 1 to about 25 mg/m², and still even more preferably about 1 to about 5 mg/m².

Typically, for therapeutic applications, the treatment would be for the duration of either conditions ITP or HIT in the subject. Further, it will be apparent to one of ordinary skill in the art that the optimal quantity and spacing of individual dosages will be determined by the nature and the severity of either ITP or HIT, the form, route and site of administration, and the nature of the particular individual being treated. Also, such optimal conditions can be determined by conventional techniques.

In many instances, it will be desirable to have several or multiple administrations of the antigen-binding fragments. In certain embodiments, a given dosage may be administered 1, 2, 3, 4, 5, 6, 7, 8, 9, 10, or more times. The dosage may be administered once to the subject, or more than once at certain interval(s), for example, once a day, once a week, twice a week, three times a week, once a month, twice a month, three times a month, once every two months, once every three months, once every six months, or once a year. The duration of treatment and any changes to the dosage and/or frequency of treatment can be altered or varied during the course of treatment in order to meet the particular needs of the subject. It will be apparent to one of ordinary skill in the art that the optimal course of treatment can be ascertained using conventional course of treatment determination tests.

In certain embodiments, the antigen-binding fragments can be formulated for various routes of administration, for example, by parenteral (e.g. intradermal, intravenous, intraspinal, intraperitoneal, subcutaneous or intramuscular), or by way of an implanted reservoir. Systemic or parenteral administration includes, but is not limited to, intraperitoneal, intramuscular, subcutaneous, intramucosal, and intravenous injections. In one embodiment, the antigen-binding fragments are administered by an intravenous route. Non-limiting examples of acceptable routes of mucosal administration including intranasal, buccal, genital tract, rectal, intratracheal, skin and the gastrointestinal tract.

In certain methods described herein, antigen-binding fragments may be administrated in combination with other agents or therapies, including administration in combination with current standard of care with anticoagulants or alternative anticoagulants. The antigen-binding fragments may be components of pharmaceutical formulations or medicaments as are described herein. Non-limiting examples of additional agents or therapies for HIT include; argatroban, bivalirudin, fondaparinux, warfarin, danaparoid, rivaroxaban, dabigatran and apixaban. Non-limiting examples of additional agents or therapies for ITP include prednisone, gamma globulin, anti-Rho(D) immune globulin (WinRho), rituximab, danazol, azathioprine, cyclophosphamide, vincristine, vinblastine and romiplostim. The antigen-binding fragments may be administered to the subject simultaneously with the additional agents or therapies. Such additional agent/s may be administered orally or by another route, for example via IV injection. Additionally, or alternatively, the antigen-binding fragments thereof may be administered to the subject before or after the additional agent/s or therapy/ies are administered.

### EXAMPLES

The present invention will now be described with reference to specific example(s), which should not be considered as limiting in any way.

### Example 1

### Materials and Methods

### Patients

Samples were selected randomly from stored sera collected with informed consent from patients with HIT. The diagnosis of HIT was made according to the criteria as previous defined (Amiral J et al. (1998) Baillieres Clin. Haematol.). The study was approved by the South-Eastern Sydney Illawarra/Eastern Sydney Area Health Service Ethics Committees. Blood and plasma from healthy donors were obtained with informed consent.

Molecular Cloning and Humanization of the scFv

Total RNA was isolated from IV.3 hybridoma cells with the RNeasy Mini Kit (Qiagen, Germany) according to the manufacturer's instructions. The first-strand cDNA was synthesized with the SuperScript™ III First-Strand Synthesis SuperMix for qRT-PCR (Invitrogen, Carlsbad, Calif.). PCR amplification of the heavy chain and light chain was conducted using specific primers. Heavy Chain Variable Region Forward Primer 5′-GGCCAGTGGATAGTCAGATGGGGGTGTCGTTTTGGC-3′(SEQ ID NO: 24) and Reverse Primer 5′-GAGGTGAAGCTGGTGGAGTC-3′ (SEQ ID NO: 25). Light Chain Variable Region Forward Primer 5′-GGATACAGTTGGTGCAGCATC-3′ (SEQ ID NO: 26) and Reverse Primer 5′-GATATTGTGATGACGCAGGCT-3′ (SEQ ID NO: 27).

For VH, the PCR was performed at 95° C. for 5 minutes, 35 cycles at 94° C. for 45 seconds, 50° C. for 45 seconds, 72° C. for 90 seconds, followed by 72° C. for 5 minutes; for VL, the conditions were 95° C. for 5 minutes, 30 cycles at 94° C. for 45 seconds, 54° C. for 45 seconds, 72° C. for 90 seconds, followed by 72° C. for 5 minutes. Both amplified fragments were cloned into PCR®-Blunt vector (Invitrogen). The nucleotide sequences of both variable fragments were confirmed with BigDye Terminator v3.1 Sequencing Reagent (Applied Biosystems, CA). The scFv was humanized by using the Complementarity Determining Region (CDR)-grafting and point mutation approach. The sequences of the six CDRs were preserved and selected amino acid residues were changed in the framework regions (FRs) to reflect the subgroups of human VH and VL domains. This was conducted with reference to the IMGT database available at http://www.biochem.unizh.ch/antibody. The sequence encoding the humanized VH and VL domains, fused with a short linker Gly₁₂-Ser₅ (SEQ ID NO: 9) was chemically synthesized (DNA2.0, Menlo Park, Calif.) with codon optimization for increased protein expression in bacteria. The constructs were cloned into pET11a expression vector and include HIS₆-tags at the C-terminus for purification. The vector was transformed into OverExpress™ CD41(DE3) competent cells (Lucigen Corporation, Middleton, Wis.) already containing the pKJE7 chaperone plasmid (Takara Bio Inc, Otsu, Japan). Transformed cells were grown in the presence of ampicillin (100 μg/ml) and chloramphenicol (20 μg/ml). The DNA of HRU2, HRU3 and HRU4 was synthesized by GenScript and the protein was expressed in OverExpress™ CD41(DE3) E. coli cells.

For CTBR1, total RNA was isolated from CTBR1 hybridoma cells with the RNeasy Mini Kit (Qiagen, Germany) according to the manufacturer's instructions and the heavy and light chain variable regions cloned as described above for IV.3.

### Protein Expression and Purification

Expression of scFvs was induced with 0.05% L-arabinose and 0.5 mM IPTG for 4 hours at 30° C. Bacterial cells were lysed in lysis buffer (50 mM Tris-Cl, 300 mM NaCl, 0.5% Triton X-100, 2 mM phenylmethanesulfonylfluoride, EDTA-free protease inhibitors (Roche Diagnostics, Castle Hill, Australia)), purified by HIS affinity chromatography and its identity confirmed by Western blot with an anti-Penta-HIS antibody. The purified scFv was examined with SDS-PAGE under reducing conditions and detected by Western Blotting with Penta-HIS antibody (Qiagen; 34660) followed by incubation with polyclonal rabbit anti-mouse immunoglobulins conjugated with horseradish peroxidase. The signal was developed with Western Lightning® Plus-ECL, Enhanced Chemiluminescence Substrate (Perkin-Elmer, Waltham, Mass., USA) and detected with ImageQuant LAS4000 imager (GE Healthcare, Uppsala, Sweden). CTBR1 was purified from hybridoma cell supernatants using protein G chromatography. CTBR1 Fab was prepared by digestion with papain agarose beads.

### Platelet Binding for Flow Cytometry Analysis

Platelet-rich plasma (PRP) was obtained from whole blood after centrifugation at 200×g for 10 minutes. Washing/Suspension buffer (PBS, 0.5% BSA, 25 mM EDTA, pH 6.8) was used for platelet preparation throughout the experiment. 4 μl of PRP was incubated with HRU molecules (2 μM) for 30 minutes. Cells were washed once with 500 μl of buffer (PBS, 0.5% BSA, 25 mM EDTA, pH 6.8) and incubated with anti-c-Myc-AF-488. After washing, platelets were suspended in 200 μl of PBS and analyzed by flow cytometry (CantoII, BD Biosciences). Anti-c-Myc-AF-488 in the absence of HRU was used as negative control.

### Platelet Aggregation

Platelet aggregation assays were performed by mixing samples at 1200 rpm, 37° C. for 15 minutes in an aggregometer (Chrono-log Aggro/Link™). The reaction mixture (500 μl) consisted of 300 μl of healthy donor PRP, HIT patient serum or purified Total HIT IgG (up to 14 μM, adjusted according to different donor platelets) and heparin (0.5 IU/ml). The degree of aggregation was determined by the increase in light transmittance. Platelet aggregation levels over 20% were considered positive. For thrombin induced platelet aggregation samples were mixed as described above in the presence of 0.3 U of thrombin.

### Serotonin Release Assay

SRA is the gold standard to confirm the presence of platelet-activating HIT antibodies [as described by Sheridan D et al. (1986) Blood]. Platelets from a healthy donor were labelled with Hydroxytryptamine Binoxalate, 5-[2-¹⁴C]-(Serotonin) (¹⁴C-5HT) (1.5 μl/ml) and mixed with heparin [0.1 (low dose) or 100 IU/ml (high dose)] in the presence of HIT serum. The protein of interest was then added to the reaction and incubated at room temperature for 1 hour. After centrifugation the supernatant was collected and the ¹⁴C-serotonin released was measured in a scintillation counter. The percentage of ¹⁴C-serotonin released was calculated by determining the proportion in the supernatant relative to the remaining ¹⁴C-serotonin in platelets. A test result is defined as positive if more than 20% serotonin release is detected with a therapeutic heparin concentration of (0.1 IU/ml) but not with a high dose of heparin (100 IU/ml).

### Microfluidics Device and Image Acquisition

Vena8 Fluoro+™ biochip micro-channels were coated with von willebrand factor (vWf) at a concentration of 200 μg/ml at 4° C. overnight. After washing with PBS, the micro-channels were blocked with PBS/1% BSA for 30 minutes. Whole blood anti-coagulated with ACD was labelled with DiOC₆ (Life Technologies) and incubated for 10 minutes at RT in the dark.

The assay was performed at 37° C. in a Venaflux micro-fluidics device (Cellix Ltd. Dublin, Ireland) in the absence or presence of HRU1 or HRU4 with patient HIT IgG or normal IgG as control at a fluid shear rate of 20 dyne/cm² for up to 460 seconds. Flow chambers were mounted on a fluorescent microscope (Zeiss Axio Observer.A1) and fluorescence images from different microscopic fields were captured in real time with a Q-Imaging EXi Blue™ camera (QImaging, Surry, BC, Canada) driven by Venaflux software (Cellix Ltd. Dublin, Ireland). The fluorescence images were analyzed with Image-Pro Premier 9.1 software (Media Cybernetics, Inc, Rockville, Md., USA). Thrombus formation was measured by calculating the thrombus mean diameter and surface area coverage.

### Thrombin Time Assay

The STA-Thrombin kit (Diagnostica STAGO S.A.A.) was used for the determination of the thrombin time by a hemostasis analyser. Plasma was prepared from ACD anticoagulated normal blood by centrifugation for 10 minutes at 2500×g. 500 μl of undiluted plasma, in the absence and presence of different inhibitor concentrations (e.g. HRU2 and HRU3), was analysed. Thrombin time of the plasma was determined by the analyser.

### Animal Model of HIT Using FcγRIIA/hPF4 Double Transgenic Mice

The mice used in these experiments were described previously (Reilly M P et al. (2001) Blood). Animals were injected intravenously (IV) with purified HIT IgG or the HIT-like MoAb KKO. Normal human IgG or an irrelevant antibody were used as controls. Heparin was injected intraperitoneally at 1 U/g following IgG injection. In some experiments HRU molecules or CTBR1 Fab were also injected via the IV route. For in vivo platelet labelling anti-CD42c-Dylight649 (Emfret) was injected IV at 1 μg/g. Platelet counts were measured before treatment and at 3 hours and 5 hours. Lungs were harvested 5-6 hours after treatment and fixed in formalin. Lungs were scanned for fluorescence in and IVIS Lumina Spectrum CT Spectrometer (PerkinElmer). For histology, lungs were processed through paraffin and sectioned. Sections were stained with hematoxylin and eosin. Other sections were stained with an anti-mouse platelet antibody and DAPI.

### Animal Model of Immune Thrombocytopenia Using FcγRIIa/hPF4 Double Transgenic Mice.

Animals were injected intraperitoneally with 0.5 μg/g or 1 μg/g of purified anti mouse GPIX antibody (Emfret). Isotype IgG (Emfret) was used as control. In some experiments, HRU1 was co-injected into the mice. Platelet counts were measured before treatment (time 0) and at 1 hour, 3 hours and 5 hours post-treatment. Lungs were harvested 5-6 hours after treatment and fixed. Lungs were scanned in and IVIS Lumina Spectrum CT Spectrometer within 2 hours of harvesting. For histology, lungs were embedded in paraffin blocks and sectioned. Sections were stained with hematoxylin and eosin. In some sections platelet rich thrombi were detected with anti-CD42c-Dylight649 (in vivo labelling). Sections were counter-stained with DAPI.

### Example 2—Sequences and Protein Expression of scFvs

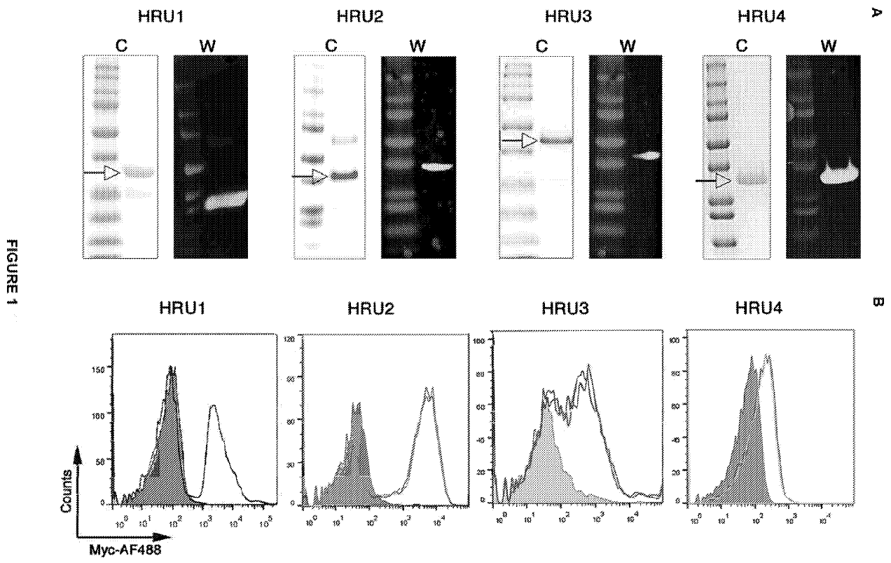

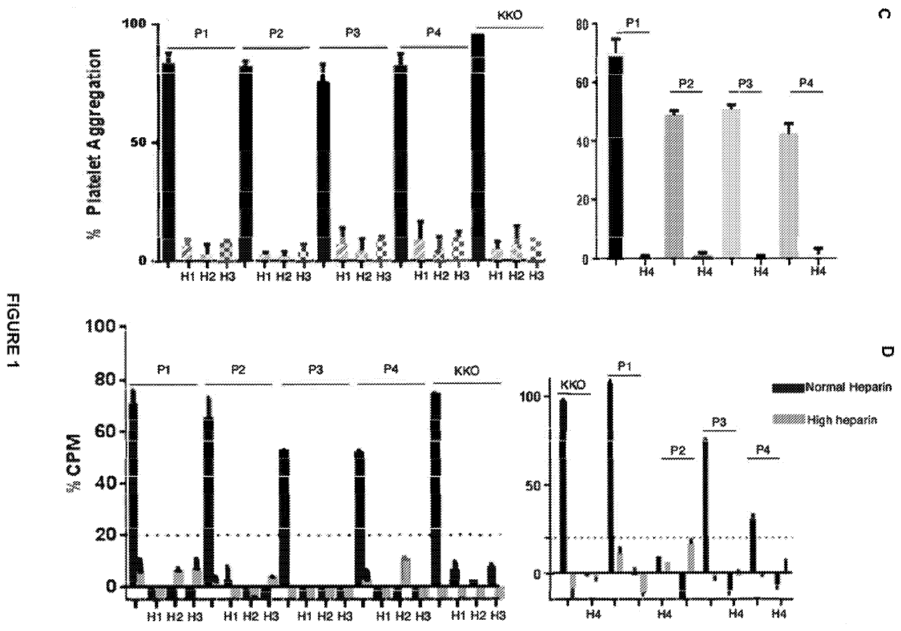

The sequences of the murine scFv as well as the humanized molecules (HRU1 to HRU4) and the variable heavy and variable light regions of CTBR1 are shown as SEQ ID NOs: 1-23 in Table 1. The purified proteins obtained from bacterial expression are shown in FIG. 1A.

TABLE 1
Sequence information

| SEQ | | |
|---|---|---|
| ID | | |
| NO: | DESCRIPTION | SEQUENCE |
| 1 | scFv murine protein | VH: |
| | sequence derived | EVKLVESGPE LKKPGETVKI SCKASGYTFT NYGMNWVKQA |
| | from IV.3 hybridoma | PGKGLKWMGW LNTYTGESIY PDDFKGRFAF SSETSASTAY |
| | cells (CDRs are | LQINNLKNED MATYFCARGD YGYDDPLDYW GQGTSVTVSS |
| | underlined) | VL: |
| | | DIVMTQAAPS VPVTPGESVS ISCRSSKSLL HTNGNTYLHW |
| | | FLQRPGQSPQ LLIYRMSVLA SGVPDRFSGS GSGTAFTLSI |
| | | SRVEAEDVGV FYCMQHLEYP LTFGAGTKLE LKRA |
| 2 | HRU1 The CDRs are | EVKLVESGPE LKKPGETVKI SCKASGYTFT NYGMNWVKQA |
| | underlined. The linker | PGKGLKWMGW LNTYTGESIY PDDFKGRFAF SLETSASTAY |
| | is in italics. Changes | LQINNLKSED TATYFCARGD YGYDDPLDYW GQGTSVTVSS |
| | between HRU1 VH | GGGGSGGGGS GGGGSDIVMT QAPPSVPVTP GESVSISCRS |
| | and VL sequences | SKSLLHTNGN TYLHWFLQRP GQSPRLLIYR MSVLASGVPD |
| | and the VH and VL | RFSGSGSGTA FTLSISRVEA EDVGVYYCMQ HLEYPLTFGA |
| | sequences of IV.3 | GTKLEIKRA |
| | scFv murine | |
| | sequences are shown | |
| | in bold. | |
| 3 | HRU1 VH-CDR1 | NYGMN |
| 4 | HRU1 VH-CDR2 | WLNTYTGESIYPDDFKG |
| 5 | HRU1 VH-CDR3 | GDYGYDDPLDY |
| 6 | HRU1 VL-CDR1 | RSSKSLLHTNGNTYLH |
| 7 | HRU1 VL-CDR2 | RMSVLAS |
| 8 | HRU1 VL-CDR3 | MQHLEYPLT |
| 9 | HRU1 Linker | SSGGGGSGGGGSGGGGS |
| 10 | HRU2 (HRU1 linked | EVKLVESGPE LKKPGETVKI SCKASGYTFT NYGMNWVKQA |
| | to bivalirudin) | PGKGLKWMGW LNTYTGESIY PDDFKGRFAF SLETSASTAY |
| | Bivalirudin sequence | LQINNLKSED TATYFCARGD YGYDDPLDYW GQGTSVTVSS |
| | is bold | SGGGGSGGGG SGGGGSDIVM TQAPPSVPVTP GESVSISCRS |
| | Linkers are in | SKSLLHTNG NTYLHWFLQR |
| | italics | PGQSPRLLIY RMSVLASGVP DRFSGSGSGT AFTLSISRVE |
| | | AEDVGVYYCM QHLEYPLTFG AGTKLEIKRA GGGGSGGGGS |
| | | GGGGFPRPGG GGNGDFEEIP EEYL |
| 11 | HRU3 (HRU1 linked | EVKLVESGP ELKKPGETVK ISCKASGYTF TNYGMNWVKQA |
| | to lepirudin) | PGKGLKWMG WLNTYTGESI YPDDFKGRFA FSLETSASTAY |
| | Lepirudin sequence is | LQINNLKSE DTATYFCARG DYGYDDPLDY WGQGTSVTVS |
| | bold | SGGGGSGGGG SGGGGSDWMT QAPPSVPVTP GESVSISCRS |
| | Linkers are in | SKSLLHTNG NTYLHWFLQR |
| | italics | PGQSPRLLIY RMSVLASGVP DRFSGSGSGT AFTLSISRVE |
| | | AEDVGVYYCM QHLEYPLTFG AGTKLEIKRA GGGGSGGGGS |
| | | GGGGLTYTDC TESGQNLCLC EGSNVCGQGN |
| | | KCILGSDGEKNQCVTGEGTP KPQSHNDGDF EEIPEEYLQ |
| 12 | HRU4 (HRU1 with | EVQLVESGP ELKKPGETVK ISCKASGYTF TNYGMNWVKQ |
| | mutations) The four | APGKGLKWMG WLNTYTGESI YPDDFKGRFA FSLETSASTA |
| | changes between | YLQINNLKSE DTATYFCARG DYGYDDPLDY WGQGTSVTVS |
| | HRU1 and HRU4 are | SGGGGSGGGG SGGGGSDIVM TQAPPSVPVT PGESVSISCR |
| | shown in bold. | SSKSLLHTNG NTYLHWFLQK |
| | Linkers are in | PGQSPRLLIY RMSVLASGVP DRFSGSGSGT DFTLKISRVE |
| | italics | AEDVGVYYCM QHLEYPLTFG AGTKLEIKRA EQKLISEEDL |
| 13 | HRU4 Heavy chain | EVQLVESGP ELKKPGETVK ISCKASGYTF TNYGMNWVKQ |
| | variable region | APGKGLKWMG WLNTYTGESI YPDDFKGRFA FSLETSASTA |
| | | YLQINNLKSE DTATYFCARG DYGYDDPLDY WGQGTSVTV |
| 14 | HRU4 Light chain | DIVM TQAPPSVPVT PGESVSISCR SSKSLLHTNG NTYLHWFLQK |
| | variable region | PGQSPRLLIY RMSVLASGVP DRFSGSGSGT DFTLKISRVE |
| | | AEDVGVYYCM QHLEYPLTFG AGTKLEIKRA EQKLISEEDL |
| 15 | CTBR1 scFv. Linkers | EVQLEESGG GLVKPGGSLK LSCAASGFTF STYAMSWVRQ |
| | are in italics. | TPEKRLEWVA TISSGGGYTY YPDSVKGRFT ISRDNAKNTL |
| | Predicted CDRs are | YLQMSSLRSE DSAMYYCARI YYYGSRNGYF DHWGQGTTLT |
| | underlined | VSSGGGGSGG GGSGGGGSDI VMTQSPASLA VSLGQRATIS |
| | | YRASKSVSTS GYSYMHWNQQ |
| | | KPGQPPRLLI YLVSNLESGV PARFSGSGSG TDFTLNIHPV |
| | | EEEDAATYYC QHIRELTRSE GGPSWKEIKR ADAAPTVSS |
| 16 | CTBR1 scFV VH- | FSTYAMS |
| | CDR1 | |
| 17 | CTBR1 scFV VH- | tissgggytyyPDSVKG |
| | CDR2 | |
| 18 | CTBR1 scFV VH- | RIYYYGSRNGYFDH |
| | CDR3 | |
| 19 | CTBR1 scFV VL- | RASKSVSTSGYSYMH |
| | CDR1 | |
| 20 | CTBR1 scFV VL- | YLVSNLES |
| | CDR2 | |
| 21 | CTBR1 scFV VL- | QHIRELTRS |
| | CDR3 | |
| 22 | CTBR1 Heavy chain | EVQLEESGG GLVKPGGSLK LSCAASGFTF STYAMSWVRQ |
| | variable region | TPEKRLEWVA TISSGGGYTY YPDSVKGRFT ISRDNAKNTL |
| | | YLQMSSLRSE DSAMYYCARI YYYGSRNGYF DHWGQGTTLT V |
| 23 | CTBR1 Light chain | DI VMTQSPASLA VSLGQRATIS YRASKSVSTS GYSYMHWNQQ |
| | variable region | KPGQPPRLLI YLVSNLESGV PARFSGSGSG TDFTLNIHPV |
| | | EEEDAATYYC QHIRELTRSE GGPSWKEIKR ADAAPTVSS |

* SEQ ID NOs: 10, 11-13, 15 and 22 may also each comprise a signal peptide sequence to assist in secretion. In the present studies, the signal peptide sequence was KSLITPITAGLLLALSQPLLA (SEQ ID NO: 28). A signal peptide sequence would not normally be present in the final protein preparation.
** SEQ ID NOs: 2 and 10-15 may each also comprise a polyhistidine tag (H₆) used for purification of the antigen-binding molecules. The final antigen-binding product may or may not comprise a polyhistidine tag.

### Example 3—HRU1 to HRU4: Inhibition of Platelet Aggregation and Activation

The capacity of HRU molecules to interact with human platelets is shown in FIG. 1B. These experiments show that all scFvs demonstrate strong binding to human platelets.

The capacity of the scFvs to inhibit platelet aggregation and activation was investigated. To ascertain whether the scFvs were capable of inhibiting HIT antibody-induced platelet aggregation in vitro, HRU1-4 was added to reactions consisting of human platelets, HIT IgG or the IgG-like monoclonal antibody KKO and heparin. In the absence of HRU molecules, HIT IgGs and KKO induced strong platelet aggregation (FIG. 1C). HRU scFvs strongly inhibited HIT IgG induced aggregation at submicromolar concentrations (2 μM in all cases). These results show that these are functional molecules able to block platelet aggregation induced by HIT IgG and heparin. The serotonin release assay (Sheridan D et al. (1986) Blood) used to measure platelet activation remains widely used as a functional assay for detection of the HIT antibodies. As shown in FIG. 1D, in the absence of HRUs, HIT IgG and low dose heparin (0.1 IU/ml) caused strong ¹⁴C-serotonin release while levels of ¹⁴C-serotonin released at high heparin concentrations were not significant (100 IU/ml) (high heparin concentrations dissociate the HIT immune complex and are used as control in these assay). When HRUs were added, however, a potent reduction in the amount of ¹⁴C-serotonin released at 0.1 IU/ml heparin was observed, indicating that these molecules are potent inhibitors of HIT IgG-induced platelet activation.

### Example 4—Anti-Thrombin Activity of HRU2 and HRU3

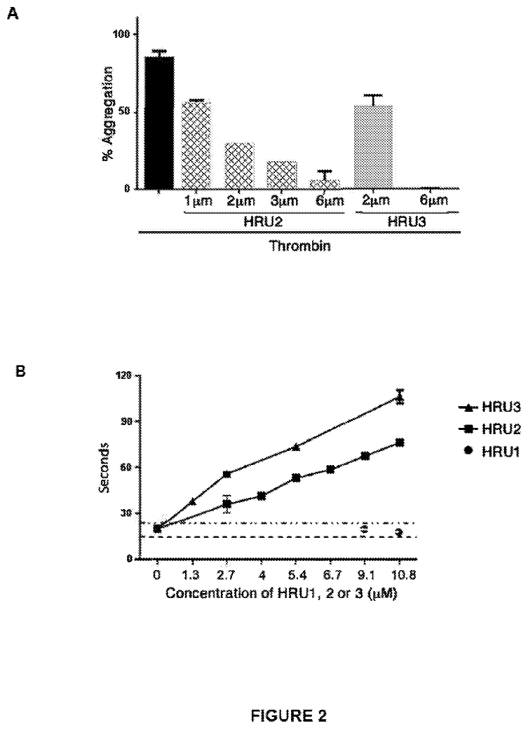

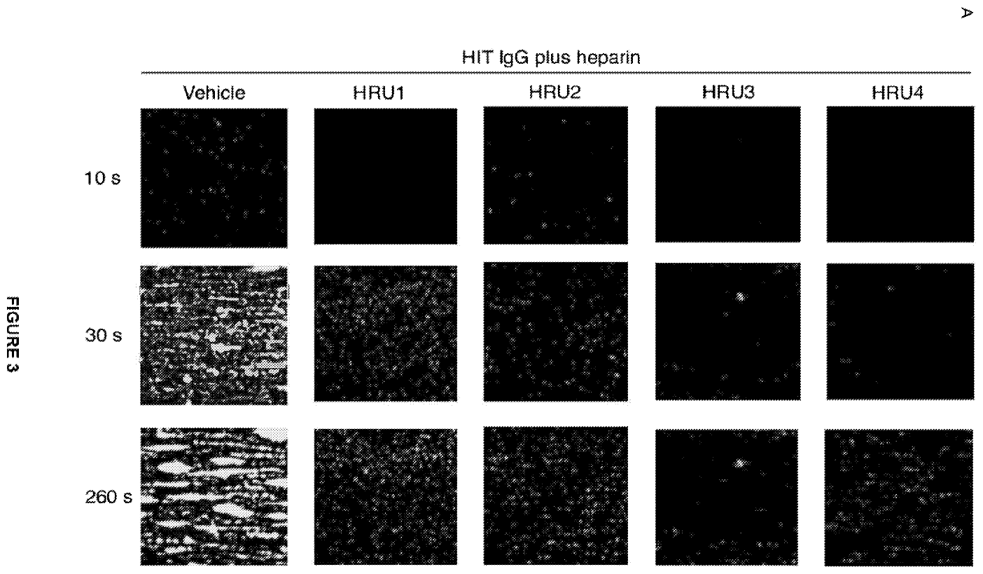

HRU2 and HRU3 were designed with dual activity: blocking FcγRIIA and directly inhibiting thrombin (with bivalirudin or lepirudin). FIG. 2A shows that both HRU2 and HRU3 inhibit thrombin-induced platelet aggregation in a dose dependent manner.

To determine the anti-clotting activity of HRU2 and HRU3 the Hemoclot thrombin inhibitor assay was used. The normal clotting time range is 15-24 seconds. Increasing concentrations of HRU2 and HRU3 (2.7-10.8 μM) resulted in significant increases in thrombin clotting time (FIG. 2B). These results indicate that HRU2 and HRU3 have effective anti-thrombin activity. Of note, HRU1 (which does not possess anti thrombin activity) does not inhibit thrombin-induced platelet aggregation (FIG. 2B).

### Example 5—Reconstitution of the HIT Condition in a Microfluidics Chamber

Thrombus formation in the circulation occurs in the context of flow-dependent contact between platelets, plasma and the vessel wall. Microfluidics devices allow quantitative evaluation of the activity of potential anti-thrombotic agents (Zwaginga J J et al. (2006) J. Thromb. Haemost.) in a system that closely imitates in vivo conditions. The HIT condition was reconstituted using whole blood from non-medicated normal donors anti-coagulated with EDTA on a Vena8 Fluoro+™ biochip coated with vWf at a shear rate of 20 dyne/cm² at 37° C.

### Example 6—HRU1 to HRU4 Inhibit Thrombus Deposition

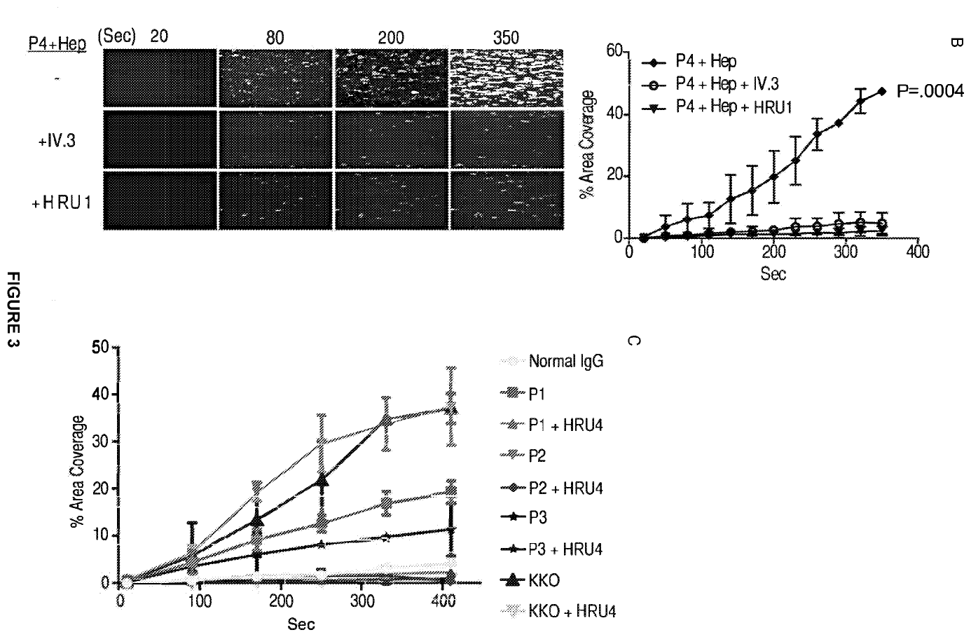

Representative microfluidics chamber images are shown in FIG. 3A. Complete inhibition of thrombus deposition was observed in the presence of HRU1-4. Percent coverage area plots show significant decrease in thrombus deposition in the presence of HRU1 (FIG. 3B) and HRU4 (FIG. 3C). Moreover, there was no detectable difference between HRU1 and the parental MoAb IV.3 in the samples under consideration (FIG. 3B). This indicates that HRU1-HRU4 are effective inhibitors of HIT Ab-induced thrombus formation under flow conditions.

### Example 7—Activity of HRUs in an Animal Model of HIT Using FcγRIIA/hPF4 Double Transgenic Mice

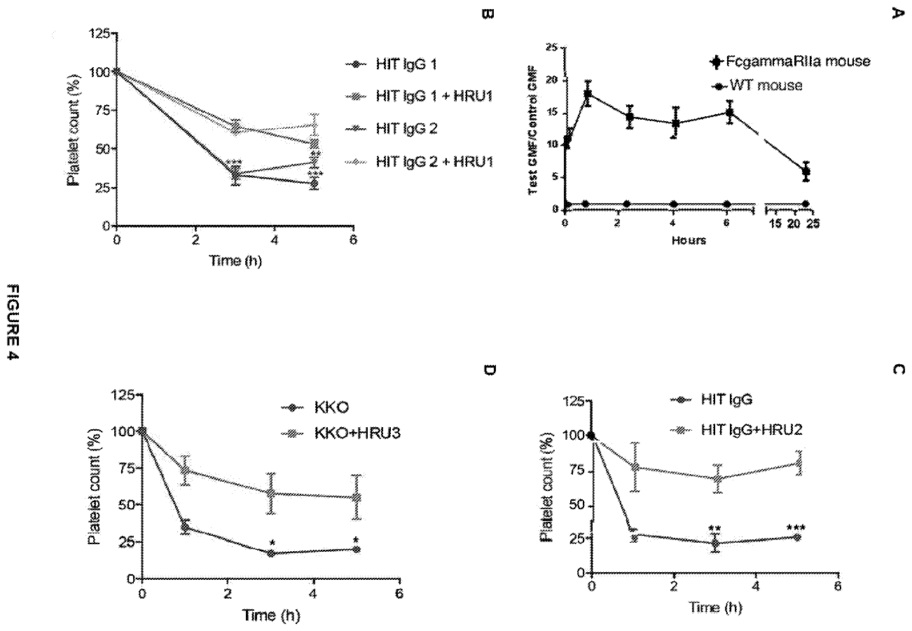

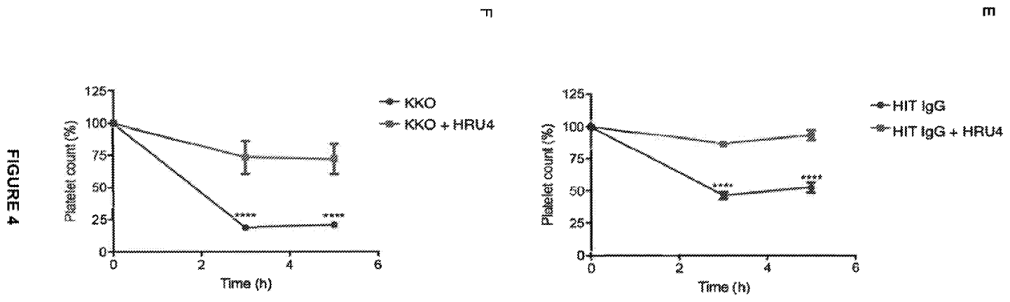

Reconstitution of the HIT condition requires expression of both human FcγRIIA and human PF4. Double transgenic mouse line (Tg mice) expressing both human proteins were used. These animals have previously been established as a model of HIT (Reilly M P et al. (2001) Blood). The graph in FIG. 4A shows the fluorescence intensity determined by flow cytometry of mouse platelets after injection of labelled HRU1. This indicates that HRU1 interacts with Tg mouse platelets in vivo. Administration of HIT IgG plus heparin induces severe thrombocytopenia in these Tg mice. Importantly, the drop-in platelet numbers was significantly arrested upon treatment with HRU1, HRU2, HRU3 or HRU4 (FIG. 4B to F), indicating that HRUs can counteract the pathogenic activity of HIT IgG in vivo. The most critical aspect of HIT is thrombosis, most commonly presented in patients as pulmonary thromboembolism. To analyze pulmonary clots in the HIT mouse model, lungs were extracted 5 hours after HIT induction, fixed and evaluated by micro CT scanning.

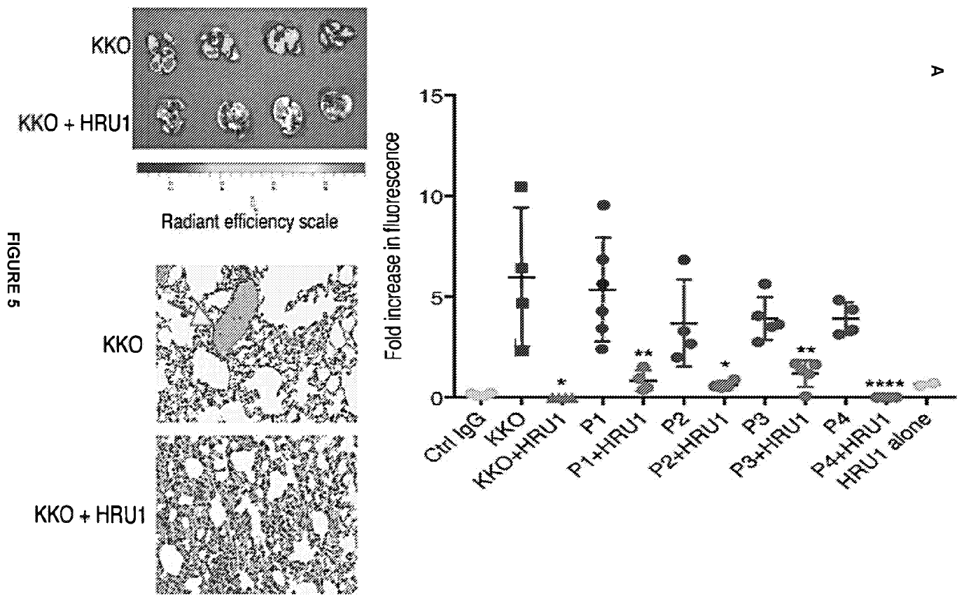

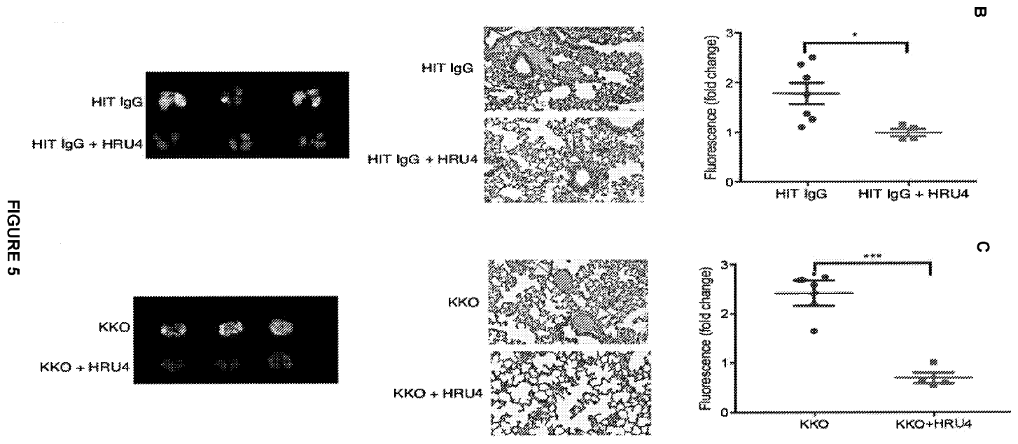

Mouse platelets were labeled in vivo with anti CD42c-Dylight649 antibody and when fixed lungs from these mice were scanned with an IVIS Lumina Spectrum CT there was clear accumulation of florescence in lungs treated with HIT IgG, indicative of the presence of platelet rich thrombi. Treatment with HRU1, HRU2, HRU3 or HRU4 resulted in a complete inhibition of thrombus accumulation (FIG. 5A-C). HRU2 and HRU3 administration als resulted in marked inhibition of thrombosis (FIGS. 5D&E). The graphs show the changes in fluorescence after HRU1, HRU2, HRU3 or HRU4 administration, which is indicative of reduction of thrombosis. Together, these data indicate that HRUs can effectively treat thrombocytopenia and thrombosis in a mouse model of HIT.

### Example 8—HRU1 Neutralizes Anti-Platelet Antibody-Induced Thrombocytopenia and Thrombosis in FcγRIIA/hPF4 Double Transgenic Mice

Anti-platelet antibodies are found in autoimmune diseases such as immune thrombocytopenia (ITP), drug-induced thrombocytopenia (DITP) and systemic lupus erythematosus (SLE). These antibodies recognise abundant platelet antigens such as the GPIbIX or GPIIbIIIa complexes. One of the mechanisms of platelet destruction in these conditions may involve engagement of the FcγRIIA receptor (Stolla M et al. (2011) Blood)

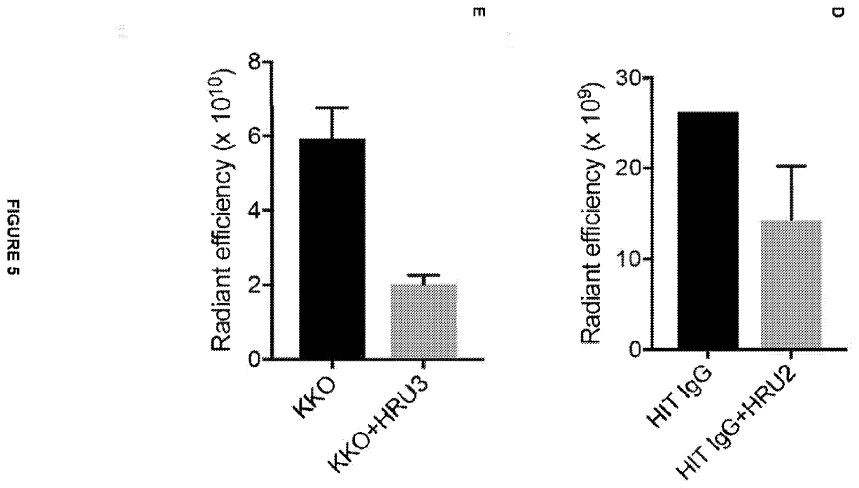

An anti-mouse GPIX antibody was tested in FcγRIIA/hPF4 Tg mice in the absence and presence of HRU1. FIG. 6A shows that this antibody induced profound thrombocytopenia in these animals. The presence of HRU4 counteracted the activity of anti-GPIX and significantly protected platelets against destruction (FIG. 6A). Analysis of extracted lungs stained with H&E demonstrated that administration of anti-GPIX antibody caused accumulation of thrombi in the lungs (FIG. 6B), however, no thrombi were detected in mice treated with HRU4 (FIG. 6B). The same results were observed in lung sections with in vivo platelet labelling using anti-CD42c-Dylight649. Platelet rich thrombi were detected only in GPIX treated mice (FIG. 6C) but not in those mice administered with HRU4. Collectively the data show that HRU1 is an effective inhibitor of thrombocytopenia and thrombosis induced by anti-platelet antibodies.

### Example 9—CTBR1

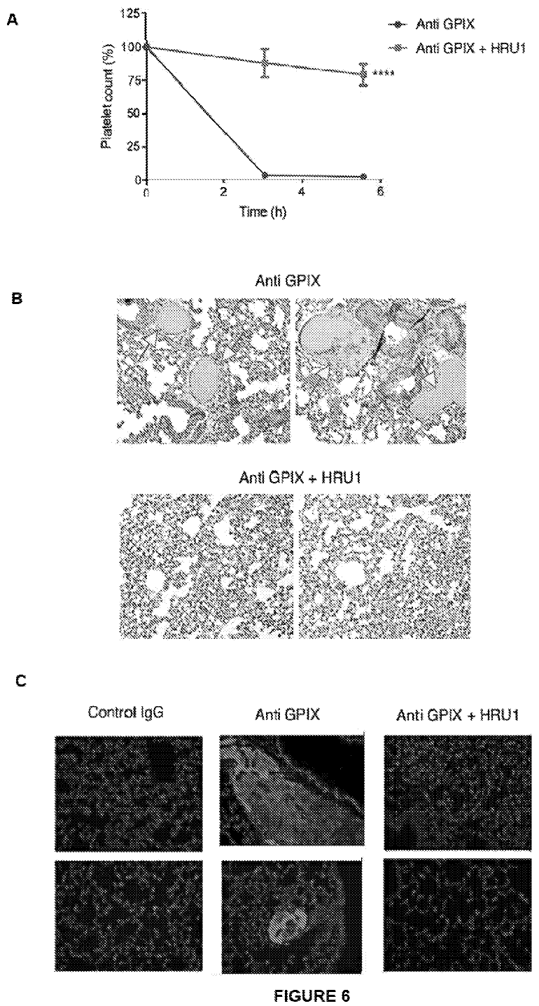

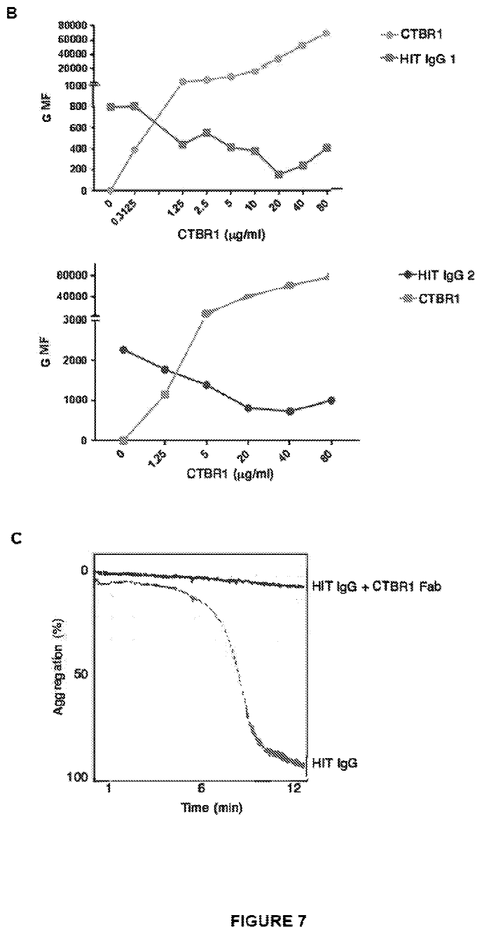

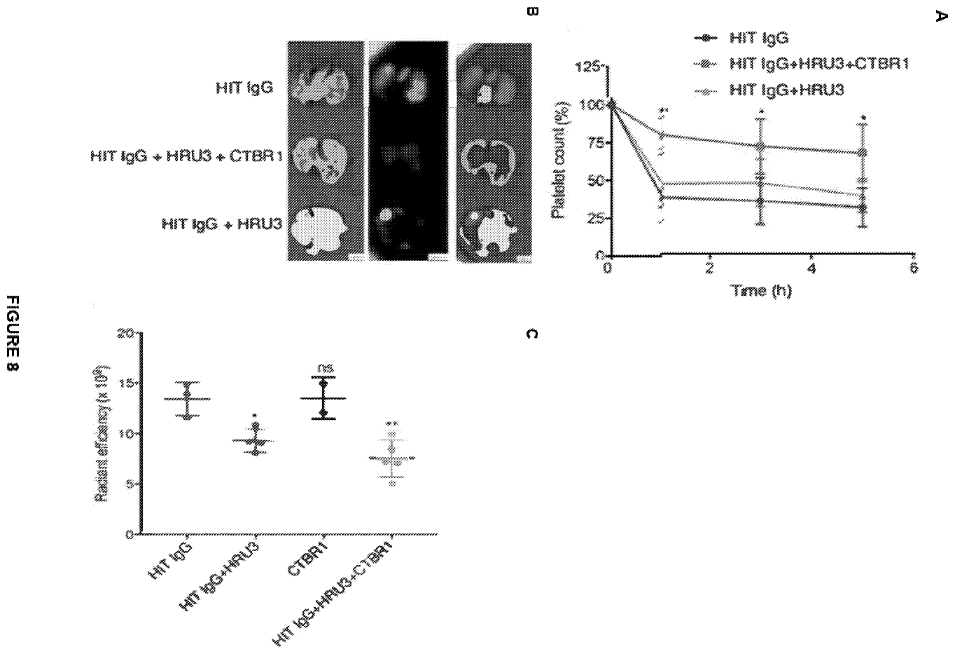

CTBR1 is a monoclonal antibody that binds to human PF4 in the absence of heparin. The Fab portion of CTBR1 binds to PF4 and competes with HIT IgG antibodies (FIGS. 7A & B). CTBR1 is therefore contemplated for use as a competitor of HIT IgGs to inhibit formation of HIT immune complexes. This in turn will inhibit HIT. FIG. 7C shows that CTBR1 Fab prevents platelet aggregation induced by HIT IgG, indicating that CTBR1 can sequester PF4 and prevent the formation of HIT immune complexes. In vivo, CTBR1 Fab acts synergistically with suboptimal concentrations of HRU3 and inhibits HIT as shown by inhibition of thrombocytopenia (FIG. 8A) and thrombosis (FIG. 8B). Together, these data show that CTBR1 can block PF4 and prevent the activity of HIT IgG antibodies.

### DISCUSSION

HIT is a serious adverse effect of heparin anticoagulant therapy and is caused by antibodies targeting the PF4/heparin immune complex. The interaction of the immune complex with the FcγRIIA on platelets is a key initiating event leading to the pathogenesis of HIT (Reilly M P et al. (2001) Blood). The current clinical management of HIT would benefit from more targeted therapies. The present studies indicate that scFvs derived from the anti-FcγRIIA MoAb IV.3 have the potential to serve as inhibitors of HIT-mediated platelet aggregation, activation, thrombocytopenia and thrombosis. The use of CTBR1 to compete with HIT antibodies binding to PF4 is also contemplated, thus preventing formation of HIT immune complexes. HRU also completely protected mice from anti-GPIX-induced thrombocytopenia and thrombosis and as such this scFv could be used to target pathogenic conditions such as ITP, DITP and SLE.

A gene construct was generated by joining the variable heavy chain and light chain domains of an anti-Fc_{γ}RIIa receptor antibody with a flexible linker. Additionally, mutational modifications were made to the framework of the scFv to obtain the best functional scFv (we termed HRU4). HRU1 was also conjugated to bivalirudin (HRU2) and lepirudin (HRU3), as bivalirudin and lepirudin are anticoagulants used in the treatment of HIT. Similar conjugation of HRU4 with anticoagulant molecule(s) is contemplated.

These constructs have been cloned into expression vectors, expressed in E. coli and HRU protein purified from bacterial lysate. Binding of HRU scFv to human platelets was detected by confocal microscopy and flow cytometry. It was shown to block strongly platelet aggregation and ¹⁴C-serotonin release induced by the HIT antibodies. In addition, HRU1, HRU2, HRU3 and HRU4 showed potent inhibition of platelet thrombus formation in a flow system using a Venaflux device.

The findings suggest that this new therapeutic approach with HRU1 or HRU4 that block the binding of HIT antibody/antigen complexes to platelet Fc_{γ}RIIa could potentially improve the treatment outcome of patients with HIT, and potential scFv conjugated with lepirudin or bivalirudin may give even better results.

The present application claims priority from Australian provisional patent application number 2019900991, the entire contents of which are incorporated herein by cross-reference.

It will be appreciated that, although specific embodiments of the invention have been described herein for the purpose of illustration, various modifications may be made without deviating from the spirit and scope of the invention as defined in the following claims.

1. An antigen-binding fragment that prevents activation of platelets by either blocking Fc_{γ}RIIa binding on platelets, neutrophils and monocytes or neutralising platelet factor 4.

2. The antigen-binding fragment of claim 1, wherein the fragment specifically binds to Fc_{γ}RIIa, and wherein the antigen-binding fragment comprises:
a heavy chain variable region comprising:
(a) a heavy chain CDR1 sequence according to SEQ ID NO: 3;
(b) a heavy chain CDR2 sequence according to SEQ ID NO: 4;
(c) a heavy chain CDR3 sequence according to SEQ ID NO: 5;
wherein said heavy chain variable region comprises at least 90% sequence identity to SEQ ID NO: 13,
and a light chain variable region comprising:
(a) a light chain CDR1 sequence according to SEQ ID NO: 6;
(b) a light chain CDR2 sequence according to SEQ ID NO: 7; and
(c) a light chain CDR3 sequence according to SEQ ID NO: 8;
wherein said light chain variable region comprises at least 90% sequence identity to SEQ ID NO: 14, and wherein the heavy chain and light chain variable regions are joined by a flexible-linker region.

3. The antigen-binding fragment of claim 1, wherein the fragment specifically binds to platelet factor 4 (PF4), and wherein the antigen-binding fragment comprises:
a heavy chain variable region comprising:
(a) a heavy chain CDR1 sequence according to SEQ ID NO: 16;
(b) a heavy chain CDR2 sequence according to SEQ ID NO: 17;
(c) a heavy chain CDR3 sequence according to SEQ ID NO: 18;
wherein said heavy chain variable region comprises at least 90% sequence identity to SEQ ID NO: 22,
and a light chain variable region comprising:
(a) a light chain CDR1 sequence according to SEQ ID NO: 19;
(b) a light chain CDR2 sequence according to SEQ ID NO: 20; and
(c) a light chain CDR3 sequence according to SEQ ID NO: 21;
wherein said light chain variable region comprises at least 90% sequence identity to SEQ ID NO: 23 and wherein the heavy chain and light chain variable regions are joined by a flexible-linker region.

4. The antigen-binding fragment according to claim 2, wherein:
(a) the heavy chain variable region comprises the amino acid sequence of SEQ ID NO:13, or a variant of that sequence having 1, 2, or 3 amino acid substitutions in the framework region; and/or
(b) the light chain variable region of SEQ ID NO:14, or a variant of that sequence having 1, 2, or 3 amino acid substitutions in the framework region.

5. The antigen-binding fragment according to claim 3, wherein:
(a) the heavy chain variable region comprises the amino acid sequence of SEQ ID NO:22, or a variant of that sequence having 1, 2, or 3 amino acid substitutions in the framework region; and/or
(b) the light chain variable region of SEQ ID NO:23, or a variant of that sequence having 1, 2, or 3 amino acid substitutions in the framework region.

6. The antigen binding fragment of claim 1, wherein the flexible-linker region is an oligopeptide having an amino acid sequence as set forth in SEQ ID NO:9.

7. An antigen-binding fragment according to claim 1, wherein the antigen-binding fragment is further conjugated to an anti-coagulant.

8. The antigen-binding fragment according to claim 7, wherein the anti-coagulant is selected from the group consisting of danaparoid, desirudin, tick anticoagulant peptide, factor Xa inhibitors, prothrombin inhibitors, tissue factor inhibitors, FXII inhibitors, danaparoid, bivalirudin, lepirudin and argatroban.

9. (canceled)

10. The antigen-binding fragment according to claim 9, which is conjugated to bivalirudin or lepirudin, and which comprises:
(i) the amino acid sequence of SEQ ID NO:10, or a variant of that sequence having 1, 2, or 3 amino acid substitutions in the framework region; or
(ii) the amino acid sequence of SEQ ID NO:11, or a variant of that sequence having 1, 2, or 3 amino acid substitutions in the framework region.

11. (canceled)

12. A nucleic acid molecule encoding an antibody-binding fragment according to claim 1, or a vector comprising the nucleic acid, or a host cell comprising the vector.

13-14. (canceled)

15. The host cell according to claim 12, wherein the host cell is derived from a mammal or insect.

16. An antigen-binding fragment according to claim 1, wherein the antigen-binding fragment is a scFv.

17. A pharmaceutical composition comprising the anti-binding fragment of claim 1.

18. A method of treating a subject with a thrombogenic-related disease, comprising administering to said subject a therapeutically effective amount of at least one antigen-binding fragment of claim 1.

19. A method of treating a subject with a disease related to Fc_{γ}RIIa-mediated neutrophil activation, comprising administering to said subject a therapeutically effective amount of at least one antigen-binding fragment of claim 1.

20. The method according to claim 18, wherein the thrombogenic-related disease is heparin-induced thrombocytopenia (HIT), immune thrombocytopenia (ITP) or immune platelet disorder with associated thrombosis, NETs-induced thrombo-embolism, organ-injury, other NETs-associated disorders, ITP associated with drugs, viral infections or antibody treatments, antiphospholipid syndrome, cancer-induced thrombocytopenia and thrombo-embolism, autoimmune or inflammatory diseases involving CD32 (including rheumatoid arthritis, osteoarthritis, systemic lupus erythematosus and psoriasis), disorders or diseases mediated by CD32 involving either one or more of the following cells: platelets, neutrophils, monocytes, macrophages, eosinophils, basophils and mast cells.

21. The method according to claim 19, wherein the ITP:
(i) is primary ITP with associated thrombosis; or
(ii) is secondary ITP with associated anti-phospholipid antibody syndrome, systemic lupus erythematosus, Evans syndrome, or chromic infection.

22. (canceled)

23. The method according to claim 18, wherein said antigen-binding fragment is administered by a route selected from the group consisting of intravenous, intramuscular, subcutaneous and intraperitoneal, or combinations thereof.

24. The method according to claim 18, wherein the therapeutically effective amount of antigen-binding fragment is from about 5 mg/kg to about 50 mg/kg.

25. The method according to claim 18, wherein the subject is human.

26-27. (canceled)
